(19) United States
(12) Patent Application Publication
(10) Pub. No.: US 2011/0020914 A1
(43) Pub. Date: Jan. 27, 2011

(54) METHODS FOR ENHANCING GROWTH OF ORGANISMS IN AN AQUEOUS GROWTH MEDIUM

(75) Inventor: Ibrahim Abou-Nemeh, Lake St. Louis, MO (US)

Correspondence Address: POLSINELLI SHUGHART PC, 700 W. 47TH STREET, SUITE 1000, KANSAS CITY, MO, 64112-1802, US

(73) Assignee: NOVUS INTERNATIONAL INC, St. Charles, MO (US)

(21) Appl. No.: 12/837,552

(22) Filed: Jul. 16, 2010

Related U.S. Application Data
(60) Provisional application No. 61/228,380, filed on Jul. 24, 2009.

(51) Int. Cl. C12N 1/12

(52) U.S. Cl. 435/257.4; 435/257.1

(57) ABSTRACT

The present invention encompasses a method for enhancing the growth, lipid content and/or protein content of bacteria, microalgae, and fungus grown in an aquatic growth medium.

### CROSS-REFERENCE TO RELATED APPLICATIONS

This application claims priority to U.S. Provisional Application No. 61/228,380 filed on Jul. 24, 2009, which is hereby incorporated by reference in its entirety.

### FIELD OF THE INVENTION

The invention encompasses methods for enhancing the growth, lipid content, and/or protein content of bacteria, microalgae, and fungus grown in an aquatic growth medium.

### BACKGROUND OF THE INVENTION

Microalgae are raised for various commercial and industrial purposes including the production of biofuels, bioplastics, pharmaceuticals, dyes, and colorants, as feedstock for other aquatic organisms such as shrimp or fish, as a food source, as a dietary supplement, and for pollution control. Microalgae obtain vital nutrients such as nitrogen, phosphorous, and micronutrients such as essential metals by absorption from the water and/or air.

Microalgae are typically raised using aquacultural methods. Using aquacultural methods, the microalgae are grown in an aquatic growth medium in which nutrients and micronutrients are dissolved in an aqueous solution. The micronutrients are typically one of the most costly and challenging elements of an aquaculture facility to manage. The availability of many of the micronutrients may be significantly influenced by the temperature and pH of the growth medium. In addition, the availability of a particular micronutrient may be influenced by the composition of the growth medium of the aquaculture facility, since chemical reactions may occur between the various dissolved ions that may precipitate a micronutrient out of solution, or dissolve a precipitate back into solution. Ideally, a growth medium should contain just enough micronutrients to provide an adequate supply to the microalgae without excessive surplus in order to minimize the cost of providing micronutrients.

Further, the growth of the microalgae is sensitive to the concentration of micronutrients in the growth medium. Although trace amounts of micronutrients are essential to the health and growth of the microalgae, an excess concentration of micronutrients may inhibit the growth of the microalgae and in extreme cases may prove fatal to the microalgae. Highly bioavailable micronutrient sources may be used in relatively low quantities in the growth medium compared to other less bioavailable sources, thereby reducing the risk of harming the microalgae due to random increases in micronutrient availability due to fluctuations in the temperature, pH, or other aspects of the growth medium during the growth cycle of the microalgae.

A need exists in the art for a method of increasing the growth of microalgae grown in an aqueous growth medium.

### SUMMARY OF THE INVENTION

One aspect of the invention comprises a method for increasing the growth of a photosynthetic microalgal cell of a colony grown in an aqueous growth medium. In particular, the method comprises contacting the growth medium with a compound of Formula (III) or with a metal salt or metal chelate of a compound of Formula (III):

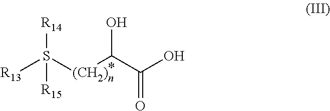

wherein:
* is a chiral carbon;
n is an integer from 1 to 3;
R₁₃ is methyl or ethyl; and
R₁₄ and R₁₅ are independently oxygen or hydrogen; wherein the growth of the microalgae is increased by at least 10% compared to the growth of microalgae grown without the compound.

In an additional aspect, the invention encompasses a method for increasing a lipid content in a photosynthetic microalgal cell of a colony grown in an aqueous growth medium. In particular, the method comprises contacting the growth medium with a compound of Formula (III) or with a metal salt or metal chelate of a compound of Formula (III):

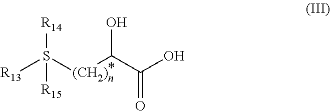

wherein:
* is a chiral carbon;
n is an integer from 1 to 3;
R₁₃ is methyl or ethyl; and
R₁₄ and R₁₅ are independently oxygen or hydrogen; wherein the amount of lipid contained in the photosynthetic microalgal cell is increased by at least 50% compared to the amount of lipid contained in the photosynthetic microalgal cell grown without the compound.

In still a further iteration, the invention provides a method for increasing a protein content in a photosynthetic microalgal cell of a colony grown in an aqueous growth medium. In particular, the method comprises contacting the growth medium with a compound of Formula (III) or with a metal salt or metal chelate of a compound of Formula (III):

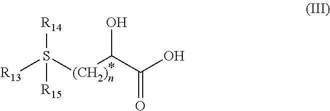

wherein:
* is a chiral carbon;
n is an integer from 1 to 3;
R₁₃ is methyl or ethyl; and
R₁₄ and R₁₅ are independently oxygen or hydrogen; wherein the amount of protein contained in the photosynthetic microalgal cell is increased by at least 500% compared to the amount of protein contained in the photosynthetic microalgal cell grown without the compound.

Other aspects and iterations of the invention are described in more detail below.

### DESCRIPTION OF FIGURES

FIG. 1 is a graph summarizing the time course of the mean cell densities of Dunaliella tertiolecta microalgal colonies cultured in the presence of five different concentrations of zinc chelated by a methionine hydroxy analog.

FIG. 2 is a graph summarizing the time course of the mean cell densities of Nitzchia sp. microalgal colonies cultured in the presence of five different concentrations of zinc chelated by a methionine hydroxy analog.

FIG. 3 is a graph summarizing the time course of the mean cell densities of Chlorella vulgaris microalgal colonies cultured in the presence of five different concentrations of BIOX-C.

FIG. 4 is a graph summarizing the time course of the mean cell densities of Chlorella vulgaris microalgal colonies cultured in the presence of five different concentrations of BIOX-M.

FIG. 5 is a graph summarizing the time course of the mean cell densities of Chlorella vulgaris microalgal colonies cultured in the presence of five different concentrations of BIOX-Z.

FIG. 6 is a graph summarizing the time course of the intracellular lipid content of Chlorella vulgaris microalgal colonies cultured in the presence of five different concentrations of BIOX-C.

FIG. 7 is a graph summarizing the time course of the intracellular lipid content of Chlorella vulgaris microalgal colonies cultured in the presence of five different concentrations of BIOX-M.

FIG. 8 is a graph summarizing the time course of the intracellular lipid content of Chlorella vulgaris microalgal colonies cultured in the presence of five different concentrations of BIOX-Z.

FIG. 9 is a graph summarizing the time course of the intracellular protein content of Chlorella vulgaris microalgal colonies cultured in the presence of five different concentrations of BIOX-C.

FIG. 10 is a graph summarizing the time course of the intracellular protein content of Chlorella vulgaris microalgal colonies cultured in the presence of five different concentrations of BIOX-M.

FIG. 11 is a graph summarizing the time course of the intracellular protein content of Chlorella vulgaris microalgal colonies cultured in the presence of five different concentrations of BIOX-Z.

### DETAILED DESCRIPTION OF THE INVENTION

It has been discovered that certain methionine compounds are effective at increasing the growth of microalgae and other unicellular organisms in an aqueous growth medium. The present invention provides a method for increasing the growth of a photosynthetic microalgae colony grown in an aqueous growth medium. The growth medium, as defined herein, refers to an aqueous solution of nutrients to be consumed by the growing microalgal colony. In particular, the method includes contacting the growth medium with an effective amount of at least one methionine compound.

### I. Methionine Compounds

One aspect of the invention encompasses compounds having at least one methionine derivative or methionine analog (hereinafter referred to as “methionine compound”).

### (a) Acyl Methionine Compounds

In an alternative embodiment, the methionine compound is an acyl methionine derivative having formula (I):

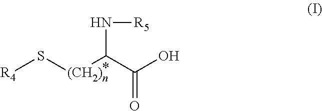

wherein:
* is a chiral carbon;
R₄ is methyl or ethyl;
R₅ is an acyl group;
n is an integer from 1 to 3.

The compound having formula (I) may be normethionine (i.e., n is 1), methionine (i.e., n is 2) or homomethionine (i.e., n is 3). Examples of suitable acyl groups (i.e., R₅) include formyl, acetyl, propionyl and succinyl. Exemplary acyl groups are formyl and acetyl. The compound having formula (I) may also be an ester derivative. Examples of suitable ester derivatives include methyl, ethyl, propyl, isopropyl, and butyl esters. For each embodiment with compounds having formula (II), both the D- and L-isomers are included within the scope of the invention. The invention also encompasses salts of compounds having formula (I). Suitable examples of salts include ammonium salts, alkaline earth metal salts (e.g., magnesium and calcium), alkali metal salts (e.g., lithium, sodium, and potassium), copper salts, zinc salts, cobalt salts, chromium salts, manganese salts, and iron salts.

In one embodiment, the compound having formula (I) is N-acetyl-L-methionine or N-acetyl-D-methionine (i.e., R₄ is methyl; R₅ is acetyl and n is 2). In another embodiment, the compound having formula (I) is N-formyl-L-methionine or N-formyl-D-methionine (i.e., R₄ is methyl; R₅ is formyl and n is 2). In still another embodiment, the compound having formula (I) is N-propionyl-L-methionine or N-propionyl-D-methionine (i.e., R₄ is methyl; R₅ is propionyl and n is 2). In a further embodiment, the compound having formula (I) is N-succinyl-L-methionine or N-succinyl-D-methionine (i.e., R₄ is methyl; R₅ is succinyl and n is 2).

### (b) Peptides Having Methionine

In a further alternative embodiment, the methionine compound may include more than one methionine amino acid residue. In this context, the methionine compound may be a peptide that includes from about 1 to about 5 methionine amino acid residues. In an additional embodiment, the methionine compound may be a peptide that has from about 2 to about 4 methionine amino acid residues. In a further embodiment, the methionine compound may be a peptide having three methionine amino acid residues. In an exemplary embodiment, the methionine compound may be a dipeptide corresponding to formula (II):

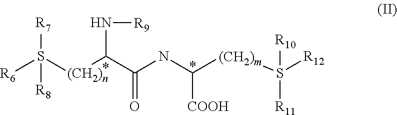

wherein:
* is a chiral carbon;
R₆ and R₁₂ are independently methyl or ethyl;
R₇, R₈, R₁₀ and R₁₁ are independently oxygen or hydrogen;
R₉ is an acyl group or hydrogen;
n is an integer from 1 to 3; and
m is an integer from 1 to 3.

Compounds corresponding to formula (II) may include one methionine sulfoxide group (e.g., one of R₇ or R₈ is hydrogen and one is oxygen) or two methionine sulfoxide groups (e.g., one of R₇ or R₈ is hydrogen and one is oxygen; and one of R₁₁ or R₁₂ is hydrogen and one is oxygen). Alternatively, compounds corresponding to formula (II) may include one methionine sulfone group (e.g., R₇ or R₈ are oxygen) or two methionine sulfone groups (e.g., R₇, R₈, R₁₀, and R₁₁ are oxygen). Depending upon the embodiment, the compound having formula (II) may include, one or two normethionines (i.e., n and/or m is 1), one or two methionines (i.e., n and/or m is 2) or one or two homomethionines (i.e., n and/or m is 3), and any combinations thereof. In certain embodiments, the compound having formula (II) may include an acyl group. Examples of suitable acyl groups include formyl, acetyl, propionyl and succinyl. Exemplary acyl groups are formyl and acetyl. The compound having formula (II) may also be an ester derivative. Examples of suitable ester derivatives include methyl, ethyl, propyl, isopropyl, and butyl esters. For each embodiment with compounds having formula (II), both the D- and L-isomers are included within the scope of the invention. In one embodiment the compound may be a D-D-isomer. In an alternative embodiment, the compound may be L-L-isomer. In a further embodiment, the compound may be a D-L-isomer. The invention also encompasses salts of compounds having formula (II). Suitable examples of salts include ammonium salt, alkaline earth metal salts (e.g., magnesium and calcium), alkali metal salts (e.g., lithium, sodium, and potassium), copper salts, zinc salts, cobalt salts, chromium salts, manganese salts, and iron salts.

### (c) Hydroxy Analogs of Methionine

In an exemplary embodiment, the methionine compound is a hydroxy analog of methionine. In one embodiment, the hydroxy analog of methionine is a compound having formula (III):

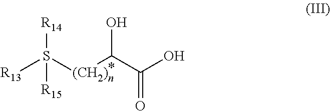

wherein:
is a chiral carbon;
n is an integer from 1 to 3;
R₁₃ is methyl or ethyl; and
R₁₄ and R₁₅ are independently oxygen or hydrogen.

Compounds corresponding to formula (III) may be a methionine sulfoxide hydroxy analog (i.e., when one of R₁₄ or R₁₅ is hydrogen and one is oxygen) or a methionine sulfone hydroxy analog (i.e., when R₁₄ and R₁₅ are oxygen). The compound having formula (III) may be an analog of normethionine (i.e., n is 1), methionine (i.e., n is 2) or homomethionine (i.e., n is 3). The compound having formula (III) may also be an ester derivative. Examples of suitable ester derivatives include methyl, ethyl, propyl, isopropyl, and butyl esters. For each embodiment with compounds having formula (III), both the D- and L-isomers are included within the scope of the invention. The invention also encompasses salts of compounds having formula (III). Suitable examples of salts include ammonium salts, alkaline earth metal salts (e.g., magnesium and calcium), alkali metal salts (e.g., lithium, sodium, and potassium), copper salts, zinc salts, cobalt salts, chromium salts, selenium salts, manganese salts, and iron salts.

In a further exemplary embodiment, the methionine compound is the hydroxy analog of methionine corresponding to formula (IV):

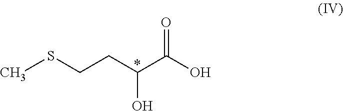

The compound having formula (IV) is 2-hydroxy-4(methylthio)-butanoic acid (commonly known as “HMTBA” and sold by Novus International, St. Louis, Mo. under the trade name ALIMET®). A variety of HMTBA salts, chelates, esters, amides, and oligomers are also suitable for use in the invention. Representative salts of HMTBA, in addition to the ones described below, include the ammonium salts, the stoichiometric and hyperstoichiometric alkaline earth metal salts (e.g., magnesium and calcium), the stoichiometric and hyperstoichiometric alkali metal salts (e.g., lithium, sodium, and potassium), and the stoichiometric and hyperstoichiometric zinc salt. Representative esters of HMTBA include the methyl, ethyl, 2-propyl, butyl, and 3-methylbutyl esters of HMTBA. Representative amides of HMTBA include methylamide, dimethylamide, ethylmethylamide, butylamide, dibutylamide, and butylmethylamide. Representative oligomers of HMTBA include its dimers, trimers, tetramers and oligomers that include a greater number of repeating units.

Alternatively, the hydroxy analog of methionine may be a metal chelate comprising one or more ligand compounds having formula (III) or formula (IV) together with one or more metal ions. Irrespective of the embodiment, suitable non-limiting examples of metal ions include zinc ions, copper ions, manganese ions, iron ions, chromium ions, selenium ions, cobalt ions, and calcium ions. In one embodiment, the metal ion is divalent. Examples of divalent metal ions (i.e., ions having a net charge of 2⁺) include copper ions, manganese ions, calcium ions, cobalt ions and iron ions. In another embodiment, the metal ion is zinc. In yet another embodiment, the metal ion is copper. In still another embodiment, the metal ion is iron. In a further embodiment, the metal ion is calcium. In each embodiment, the ligand compound having formula (III) or formula (IV) is preferably HMTBA. In one exemplary embodiment, the metal chelate is HMTBA-Ca. In a further exemplary embodiment, the metal chelate is HMTBA-Cu. In an alternative exemplary embodiment, the metal chelate is HMTBA-Zn. In still another exemplary embodiment, the metal chelate is HMTBA-Fe.

As will be appreciated by a skilled artisan, the ratio of ligands to metal ions forming a metal chelate compound can and will vary. Generally speaking, where the number of ligands is equal to the charge of the metal ions, the charge of the molecule is typically net neutral because the carboxy moieties of the ligands having formula (III) or formula (IV) are in deprotonated form. By way of further example, in a chelate species where the metal ion carries a charge of 2⁺ and the ligand to metal ion ratio is 2:1, each of the hydroxy groups is believed to be bound by a coordinate covalent bond to the metal while an ionic bond exists between each of the carboxylate groups and the metal ion. This situation exists, for example, where divalent zinc, copper, or manganese is complexed with two HMTBA ligands. By way of further example, where the number of ligands exceeds the charge on the metal ion, such as in a 3:1 chelate of a divalent metal ion, the ligands in excess of the charge generally remain in a protonated state to balance the charge. Conversely, where the positive charge on the metal ion exceeds the number of ligands, the charge may be balanced by the presence of another anion, such as, for example, chloride, bromide, iodide, bicarbonate, hydrogen sulfate, and dihydrogen phosphate.

Generally speaking, a suitable ratio of ligand to metal ion ranges from about 1:1 to about 3:1 or higher. In another embodiment, the ratio of ligand to metal ion ranges from about 1.5:1 to about 2.5:1. Within a given mixture of metal chelate compounds, the mixture will include compounds having different ratios of ligand to metal ion. For example, a composition of metal chelate compounds may have species with ratios of ligand to metal ion that include 1:1, 1.5:1, 2:1, 2.5:1, and 3:1.

Metal chelate compounds of the invention may be made in accordance with methods generally known in the art, such as described in U.S. Pat. Nos. 4,335,257 and 4,579,962, which are both hereby incorporated by reference in their entirety. Alternatively, the metal chelate compounds may be purchased from a commercially available source. For example, HMTBA-Zn and HMTBA-Cu may be purchased from Novus International, Saint Louis, Mo., sold under the trade names MINTREX® Zn and MINTREX® Cu, respectively.

In an alternative exemplary embodiment, the hydroxy analog of methionine may be a metal salt comprising an anionic compound having formula (III) or formula (IV) together with a metal ion. Typically, suitable metal ions will have a 1+, 2⁺ or a 3⁺ charge and will be selected from zinc ions, copper ions, manganese ions, selenium ions, iron ions, chromium ions, silver ions, cobalt ions, and silver ions. Without being bound by any particular theory, however, it is generally believed that combinations of zinc, copper, manganese, iron, selenium, chromium, nickel, and cobalt ions together with HMTBA form metal chelates as opposed to salts. Irrespective of whether the molecule formed is a salt or a chelate, both forms of the molecules are included within the scope of the invention. Salts useful in the invention may be formed when the metal, metal oxide, metal hydroxide or metal salt (e.g., metal carbonate, metal nitrate, or metal halide) react with one or more compounds having formula (III) or formula (IV). In an exemplary embodiment, the compound having formula (III) or formula (IV) is HMTBA.

Salts may be prepared according to methods generally known in the art. For example, a metal salt may be formed by contacting HMTBA with a metal ion source. In one embodiment, a silver ion having a 1⁺ charge may be contacted with HMTBA to form a silver 2-hydroxy-4-methylthiobutanoate metal salt. This salt generally will have a silver to HMTBA ratio of approximately 1:1.

### (d) Other Methionine Analogs

In one further exemplary embodiment, the methionine compound is an other methionine analog corresponding to formula (V):

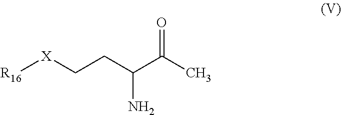

wherein:
R₁₆ is hydrogen or methyl; and
X is selenium, sulfur, carbon, or isopropyl.

Compounds corresponding to formula (V) may include selenomethionine (e.g. X is selenium and R₁₆ is a methyl group), ethionine (e.g., X is sulfur and R₁₆ is an ethyl group), norleucine (e.g. X is carbon and R₁₆ is a methyl group), or norvaline (e.g. X is an isopropyl group and R₁₆ is hydrogen).

### II. Unicellular Organisms

One aspect of the invention encompasses increasing the growth of colonies of unicellular organisms growing in suspension in a growth medium by contacting the growth medium of the colony with an effective amount of a methionine compound. A colony, as defined herein, includes a group of unicellular organisms, typically of the same species, growing within a growth medium. In various embodiments the unicellular organisms may include photosynthetic or heterotrophic algae, bacteria, and yeast, described below.

### (a) Photosynthetic or Heterotrophic Microalgae

A variety of photosynthetic microalgal species may be used in the methods of the present invention. The microalgal cells typically include a single species of single-celled aquatic algae. The microalgal species may include, but are not limited to, freshwater benthic, epiphytic, and planktonic species (inhabiting flowing sources, alkaline creeks, eutrophic lakes and ponds, oligotrophic lakes and ponds, or alpine lakes and ponds), marine algae (including tropic, temperate and cold water variants of benthic, epiphytic and planktonic habitats, living in near shore, in open ocean, in brackish water or in halophilic environments), and non-aquatic microalgae such as Antarctic algae, desert-dwelling algae, and soil algae, as well as species of unusual habitat such as ectoparasitic algae and extremophilic algae from hot springs, snow and other extreme environments.

Photosynthetic microalgal cells typically contain chloroplasts that include chlorophyll a, chlorophyll b, chlorophyll c, chlorophyll d, and combinations thereof. In addition, the photosynthetic microalgal cells may include accessory pigments including phycobilins, carotenoids, and combinations thereof. The cells of the photosynthetic and heterotrophic microalgae further include proteins, carbohydrates, and oils.

Non-limiting examples of photosynthetic and heterotrophic microalgal species that may be employed in the present invention include Charophyta, such as Zygenematophyceae (which includes Actinotaenium, Arthrodesmus, Bambuisina, Closterium, Cosmarium, Cosmocladium, Desmidium, Euastrum, Genicularia, Gonatozygon, Heimansia, Hyalotheca, Mesotaenium, Micrasterias, Mougeotia; Netrium, Onychonema, Penium, Phymatodocis, Pleurotaenium, Roya, Sphaerozosma, Spirogyra, Spondylosium, Staurastrum, Staurodesmus, Teilingia, Triploceras, Xanthidium, Zygnema, Zygogonium), Chlorokybophyceae, (which includes Chlorokybus), Mesostigmatophyceae (which includes Chaetosphaeridium and Mesostigma), Coleochaetophyceae (which includes Coleochaete) and Klebsormidiophyceae (which includes Klebsormidium); Chlorophyta, such as Chlorophyceae (which includes Acetabularia, Acicularia, Actinochloris, Amphikrikos, Anadyomene, Ankistrodesmus, Ankyra, Aphanochaete, Ascochloris, Asterococcus, Asteromonas gracilis, Astrephomene, Atractomorpha, Axilococcus, Axilosphaera, Basichlamys, Basicladia, Binuclearia, Bipedinomonas, Blastophysa, Boergesenia, Boodlea, Borodinella, Borodinellopsis, Botryococcus, Brachiomonas, Bracteacoccus, Bulbochaete, Caespitella, Capsosiphon, Carteria, Centrosphaera, Chaetomorpha, Chaetonema, Chaetopeltis, Chaetophora, Chalmasia, Chamaetrichon, Characiochloris, Characiosiphon, Characium, Chlamydella, Chlamydobotrys, Chlamydocapsa, Chlamydomonas, Chlamydopodium, Chloranomala, Chlorochydridion, Chlorochytrium, Chlorocladus, Chlorocloster, Chlorococcopsis, Chlorococcum, Chlorogonium, Chloromonas, Chlorophysalis, Chlorosarcina, Chlorosarcinopsis, Chlorosphaera, Chlorosphaeropsis, Chlorotetraedron, Chlorothecium, Chodatella, Choricystis, Cladophora, Cladophoropsis, Cloniophora, Closteriopsis, Coccobotrys, Coelastrella, Coelastropsis, Coelastrum, Coenochloris, Coleochlamys, Coronastrum, Crucigenia, Crucigeniella, Ctenocladus, Cylindrocapsa, Cylindrocapsopsis, Cylindrocystis, Cymopolia, Cystococcus, Cystomonas, Dactylococcus, Dasycladus, Deasonia, Derhesia, Desmatractum, Desmodesmus, Desmotetra, Diacanthos, Dicellula, Dicloster, Dicranochaete, Dictyochloris, Dictyococcus, Dictyosphaeria, Dictyosphaerium, Didymocystis, Didymogenes, Dilabifilum, Dimorphococcus, Diplosphaera, Drapamaldia, Dunaliella, Dysmorphococcus, Echinocoleum, Elakatothrix, Enallax, Entocladia, Entransia, Eremosphaera, Ettlia, Eudorina, Fasciculochloris, Femandinella, Follicularia, Fottea, Franceia, Friedmannia, Fritschiella, Fusola, Geminella, Gloeococcus, Gloeocystis, Gloeodendron, Gloeomonas, Gloeotila, Golenkinia, Gongrosira, Gonium, Graesiella, Granulocystis, Oocystis, Granulocystopsis, Gyorffiana, Haematococcus, Hazenia, Helicodictyon, Hemichloris, Heterochlamydomonas, Heteromastix, Heterotetracystis, Hormidiospora, Hormidium, Hormotila, Hormotilopsis, Hyalococcus, Hyalodiscus, Hyalogonium, Hyaloraphidium, Hydrodictyon, Hypnomonas, Ignatius, Interfilum, Kentrosphaera, Keratococcus, Kermatia, Kirchneriella, Koliella, Lagerheimia, Lautosphaeria, Leptosiropsis, Lobocystis, Lobomonas, Lola, Macrochloris, Marvania, Micractinium, Microdictyon, Microspora, Monoraphidium, Muriella, Mychonastes, Nanochlorum, Nautococcus, Neglectella, Neochloris, Neodesmus, Neomeris, Neospongiococcum, Nephrochlamys, Nephrocytium, Nephrodiella, Oedocladium, Oedogonium, Oocystella, Oonephris, Ourococcus, Pachycladella, Palmella, Palmellococcus, Palmellopsis, Palmodictyon, Pandorina, Paradoxia, Parietochloris, Pascherina, Pauilschulzia, Pectodictyon, Pediastrum, Pedinomotias, Pedinopera, Percursaria, Phacotus, Phaeophila, Physocytium, Pilina, Planctonenma, Planktosphaeria, Platydorina, Platymonas, Pleodorina, Pleurastrum, Pleurococcus, Ploeotila, Polyedriopsis, Polyphysa, Polytoma, Polytomella, Prasinocladus, Prasiococcus, Protoderma, Protosiphon, Pseudendocloniopsis, Pseudocharacium, Pseudochlorella, Pseudochlorococcum, Pseudococconyxa, Pseudodictyosphaerium, Pseudodidymocystis, Pseudokirchneriella, Pseudopleurococcus, Pseudoschizomeris, Pseudoschroederia, Pseudostichococcus, Pseudotetracystis, Pseudotetraëdron, Pseudotrebouxia, Pteromonas, Pulchrasphaera, Pyramimonas, Pyrobotrys, Quadrigula, Radiofilum, Radiosphaera, Raphidocelis, Raphidonenia, Raphidonemopsis, Rhizoclonium, Rhopalosolen, Saprochaete, Scenedesnius, Schizochlamys, Schizomeris, Schroederia, Schroederiella, Scotiellopsis, Siderocystopsis, Siphonocladus, Sirogonium, Sorastrum, Spermatozopsis, Sphaerella, Sphaerellocystis, Sphaerellopsis, Sphaerocystis, Sphaeroplea, Spirotaenia, Spongiochloris, Spongiococcum, Spongiococcuni, Stephanoptera, Stephanosphaera, Stigeoclonium, Struvea, Tetmemorus, Tetrabaena, Tetracystis, Tetradesmus, Tetraedron, Tetrallantos, Tetraselnis, Tetraspora, Tetrastrum, Treubaria, Triploceros, Trochiscia, Trochisciopsis, Ulva, Uronema, Valonia, Valoniopsis, Ventricaria, Viridiella, Vitreochlamys, Volvox, Volvulina, Westella, Willea, Wislouchiella, Zoochlorella, Zygnemopsis, Spermatozopsis, Hyalotheca, Pleurastrum, Chlorococcuni, Chlorella, Pseudopleurococcum, Coelastrum and Rhopalocystis), Ulvophyceae (which includes Acrochaete, Bryopsis, Cephaleuros, Chlorocystis, Enteromorpha, Gloeotilopsis, Halochlorococcum, Ostreobium, Pirula, Pithophora, Planophila, Pseudendoclonium, Trentepohlia, Trichosarcina, Ulothrix, Bolbocoleon, Chaetosiphon, Eugomontia, Oltniannsiellopsis, Pringsheimiella, Pseudodendroclonium, Pseuduilvella, Sporocladopsis, Urospora and Wittrockiella), Trebouxiophyceae (which includes Apatococcus, Asterochloris, Auxetlochlorella, Chlorella, Coccomyxa, Desmococcus, Dictyochloropsis, Elliptochloris, Jaagiella, Leptosira, Lobococcus, Makinoella, Microthamnion, Myrmecia, Nannochloris, Oocystis, Prasiola, Prasiolopsis, Prototheca, Stichococcus, Tetrachlorella, Trebouxia, Trichophilus, Watanabea and Myrmecia), Prasiniophyceae (which includes Bathycoccus, Mantonielia, Micromonas, Nephroselmis, Pseudoscourfieldia, Scherifelia, Picocystis, Pterosperma and Pycnococcus) and Charophyceans (which includes Zygogonium); Diatoms, such as Bolidophyceae (which includes Bolidomonas, Chrysophyceae, Giraudyopsis, Glossomastix, Chromophyton, Chrysamoeba, Chrysochaete, Chrysodidymus, Chrysolepidomonas, Chrysosaccus, Chrysosphaera, Chrysoxys, Cyclonexis, Dinobryon, Epichrysis, Epipyxis, Hibberdia, Lagynion, Lepochromulina, Monas, Monochrysis, Paraphysomonas, Phaeoplaca, Phaeoschizochlamys, Picophagus, Pleurochrysis, Stichogloea and Uroglena), Coscinodiscophyceae (which includes Bacteriastrum, Bellerochea, Biddulphia, Brockmanniella, Corethron, Coscinodiscus, Eucampia, Extubocellulus, Guinardia, Helicotheca, Leptocylindrus, Leyanella, Lithodesmium, Melosira, Minidiscus, Odontella, Planktoniella, Porosira, Proboscia, Rhizosolenia, Stellarima, Thalassionema, Bicosoecid, Symbiomonas, Actinocyclus, Amphora, Arcocellulus, Detonula, Diatoma, Ditylum, Fragilariophyceae, Asterionellopsis, Delphineis, Grammatophora, Nanofrustulum, Synedra and Tabularia), Dinophyceae (which includes Adenoides, Alexandrium, Amphidinium, Ceratium, Ceratocorys, Coolia, Crypthecodinium, Exuviaella, Gambierdiscus, Goniyaulax, Gymnodinium, Gyrodinium, Heterocapsa, Katodinium, Lingulodinium, Pfiesteria, Polarella, Protoceratium, Pyrocystis, Scrippsiella, Symbiodinium, Thecadinium, Thoracosphaera and Zooxanthella) and Alveolates (which includes Cystodinium, Glenodinium, Oxyrrhis, Peridinium, Prorocentrum and Woloszynskia); Rhodophyta, such as Rhodophyceae (which includes Acrochaetium, Agardhiella, Antithamnion, Antithamnionella, Asterocytis, Audouinella, Balbiania, Bangia, Batrachospermum, Bonnemaisonia, Bostrychia, Callithamnion, Caloglossa, Ceramium, Champia, Chroodactylon, Chroothece, Compsopogon, Compsopogonopsis, Cumagloia, Cyanidium, Cystoclonium, Dasya, Digenia, Dixoniella, Erythrocladia, Erythrolobas, Erythrotrichia, Flintiella, Galdieria, Gelidium, Glaucosphaera, Goniotrichum, Gracilaria, Grateloupia, Griffithsia, Hildenbrandia, Hymenocladiopsis, Hypnea, Laingia, Membranoptera, Myriogramme, Nenalion, Nemalionopsis, Neoagardhiella, Palmaria, Phyllophora, Polyneura, Polysiphonia, Porphyra, Porphyridium, Pseudochantransia, Pterocladia, Pugetia, Rhodella, Rhodochaete, Rhodochorton, Rhodosorus, Rhodospora, Rhodymrenia, Seirospora, Selenastrum, Porphyra, Sirodotia, Solieria, Spermothamnion, Spyridia, Stylonema, Thorea, Trailiella and Tuomeya); Cryptophyta, such as Cryptophyceae (which includes Campylomonas, Chroomonas, Cryptochrysis, Cryptomonas, Guillardia, Hanusia, Hemiselmis, Plagioselmis, Proteomonas, Pyrenomonas, Rhodomonas and Stroreatula); Chlorarachniophyta, such as Chlorarachnion, Lotharella and Chattonella; Haptophyta, such as Pavlovophyceae (which includes Apistonema, Chrysochromulina, Coccolithophora, Corconitochrysis, Cricosphaera, Diacronema, Emiliana, Pavlova and Ruttnera) and Prymnesiophyceae (which includes Cruciplacolithus, Prymnnesium, Isochrysis, Calyptrosphaera, Chrysotila, Coccolithus, Dicrateria, Heterosigma, Hymenomonas, Imantonia, Gephyrocapsa, Ochrosphaera, Phaeocystis, Platychrysis, Pseudoisochrysis, Syracosphaera and Pleurochrysis); Euglenophyta, such as Euglenophyceae (which includes Colacium, Euglena, Eutreptia, Eutreptiella, Lepocinclis, Phacus, Tetruetreptia and Trachelomonas); and Heterokonta, such as Phaeophyceae (which includes Ascoseira, Asterocladon, Bodanella, Desmarestia, Dictyocha, Dictyota, Ectocarpus, Halopteris, Heribaudiella, Pleurocladia, Porterinema, Pylaiella, Sorocarpus, Spermatochnus, Sphacelaria and Waerniella), Pelagophyceae (which includes Aureococcus, Aureoumbra, Pelagococcus, Pelagomonas, Pulviniaria and Sarcinochrysis), Xanthophyceae (which includes Asterosiphon, Botrydiopsis, Botrydium, Bumilleria, Bumilleriopsis, Characiopsis, Chlorellidium, Chlorobotrys, Goniochloris, Heterococcus, Heterothrix, Heterotrichella, Mischococcus, Ophiocytium, Pleurochloridella, Plenrochloris, Pseudobumilleriopsis, Sphaerosorus, Tribonema, Vaucheria and Xanthonema), Eustigmatophyceae (which includes Chloridella, Ellipsoidion, Eustigmatos, Monodopsis, Monodus, Nannochloropsis, Polyedriella, Pseudocharaciopsis, Pseudostaurastrum and Vischeria), Syanurophyceae (which includes Mallomonas, Synura and Tessellaria), Phaeothamniophyceae (which includes Phaeobotrys and Phaeothamnion) and Raphidophyceae, (which includes Olisthodiscus, Vacuolaria and Fibrocapsa).

### (b) Bacteria

In another embodiment, a variety of bacteria may be used in the methods of the present invention. Non-limiting species of bacteria that may be employed in the present invention include Acetobacter aurantius, Acinetobacter baumannii, Actinomyces lsraelii, Agrobacterium radiobacter, Agrobacterium tumefaciens, Azorhizobium caulinodans, Azotobacter vinelandii, Anaplasma phagocytophilum, Anaplasma marginale, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus, Bartonella henselae, Bartonella Quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitides, Pasteurella multocida, Pasteurella tularensis, Porphyromonas gingivalis, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Stayyereyofhia mioms, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, species from the genus Wolbachia, Yersinia enterocolitica, Yersinia pestis, and Yersinia pseudotuberculosis.

### (c) Fungus

A variety of species of fungus may be used in the methods of the present invention. The term “fungus”, as defined herein, refers to any of the organisms classified within the biological kingdom fungi, including but not limited to organisms classified within the phyla Blastocladiomycota; Chytridiomycota; Glomeromycota; Microsporidia; Neocallimastigomycota; Ascomycota including the subphyla Pezizomycotina, Saccharomycotina, and Taphrinomycotina; Basidiomycota including the subphyla Agaricomycotina, Pucciniomycotina, and Ustilaginomycotina; the sub-phylum Entomophthoromycotina; the fungal group Kickxellomycotina; the fungal group Mucoromycotina, and the fungal group Zoopagomycotina.

A variety of species of fungus may be used in the methods of the present invention, including but not limited to those species used commercially for single-cell protein production such as Aspergillus oryzae, Sclerotium rolfsii, Polyporus species, Trichoderma species including Trichoderma hazianamum, Scytalidium acidophilum, Fusarium graminearum, Penicillium cyclopium, Paeecilomyces varioti, and Chaetomium cellulolyticum.

In addition, a variety of yeast species may be used in the methods of the present invention. The term “yeast”, as defined herein, refers to a subset of unicellular fungus species that are characterized by asexual reproduction by budding or binary fission. Various species of yeast have been used domestically and commercially throughout history for various useful purposes such as alcohol fermentation, and the leavening of baked goods. Species of yeast suitable for the methods of the present invention, include but are not limited to food-grade or edible yeasts, species used for the brewing of fermented beverages, species used for single-cell protein production, and species used for large-scale ethanol production. Non-limiting examples of food-grade or edible yeasts include Saccharomyces cerevisae, Saccharomyces boulardii, Saccharomyces torula, Saccharomyces exiguous, Candida stellata, Schizosaccharomyces pombe, Torulaspora delbrueckii, and Zygosaccharomyces bailii. Yeast species typically used for the brewing of fermented beverages include but are not limited to Brettanomyces lambicus, Brettanomyces bruxellensis, Brettanomyces claussenii, and Saccharomyces pastorianus. In addition, genetically engineered yeast strains typically used for large-scale ethanol production including but not limited to various engineered strains of Saccharomyces species.

### III. Growth Medium Compositions

The growth medium of the present invention is an aqueous solution that includes dissolved macronutrients and micronutrients, in addition to the effective amount of methionine compound. The composition of the growth medium may vary depending on the particular requirements of the species that is grown in the growth medium. For example, a marine microalgal species such as a diatom species may utilize a growth medium with a much higher salinity than the growth medium used to grow fresh-water microalgal species.

### (a) Macronutrients

In one embodiment, the growth medium includes three primary macronutrients: nitrogen, phosphorus, and potassium. These macronutrients are typically consumed in relatively large quantities by the microalgae; microalgal cells typically incorporate each of the macronutrients in quantities on the order of at least 0.1% or more of the dry weight of the microalgal cells. In addition, the growth medium may provide secondary nutrients such as calcium, sulfur, or magnesium.

Naturally occurring fertilizers may be dissolved into the growth medium as a source of macronutrients. Non-limiting examples of naturally occurring fertilizers include manure, slurry, worm castings, peat, seaweed, sewage, mine rock phosphate, sulfate of potash, limestone, and guano.

Non-limiting examples of other compounds containing nutrients that may be dissolved into the growth medium include ammonia, ammonium, nitrate, urea, phosphate, and combinations thereof. Other non-limiting examples of macronutrient-containing compounds suitable for use in the growth medium include ammonium chloride, ammonium sulfate, mono-ammonium phosphate, diammonium phosphate, ammonium nitrate, sodium nitrate, potassium nitrate, calcium phosphate, super phosphate, triple super phosphate, and potassium chloride.

### (b) Micronutrients

In one embodiment, the growth medium includes micronutrients, defined herein as nutrients essential to the growth of the microalgal colony that may be included in the growth medium in relatively small or trace quantities. Non-limiting examples of suitable micronutrients for the growth medium include elements such as calcium, magnesium, sulfur, iron, copper, manganese, barium, zinc, chlorine, vanadium, selenium, sodium, molybdenum or any other element that may be beneficial to the growth of the microalgal colony.

### (c) Other Compounds

The growth medium may further include other compounds depending on the nutritional needs of the microalgal colony. For example, sodium chloride may be incorporated into the growth medium to adjust the salinity of the growth medium to within a suitable range for the species of microalgae in the colony. In another example, silica may be included in the growth media when the species of microalgae in the colony is a species of diatom that incorporates significant amounts of silica into its cell walls.

Other non-limiting examples of compounds that may be included in the growth medium include vitamins, fungicides, bactericides, herbicides, and insecticides.

i. Vitamins

The growth medium may further include vitamins depending on the nutritional needs of the particular species of the microalgal colony. Non-limiting examples of vitamins that may be included in the growth medium include Vitamin B₁, Vitamin B₂, Vitamin B₃, Vitamin B₅, Vitamin B₆, Vitamin B₇, Vitamin B₉, Vitamin B₁₂, and Vitamin C. Non-limiting examples of Vitamin B₁ include thiamine hydrochloride, thiamine nitrate, and thiamine. Non-limiting examples of Vitamin B₂ include riboflavin and its phosphates. Non-limiting examples of Vitamin B₃ include niacin and nicotinamide. Non-limiting examples of Vitamin B₅ include pantothenic acid, calcium pantothenoate, and panthenol. Non-limiting examples of Vitamin B₆ include pyridoxine, pyridoxamine, and pyridoxal. Non-limiting examples of Vitamin B₇ include biotin. Non-limiting examples of Vitamin B₉ include folic acid, folinic acid, tetrahydrofolic acid, 5-methyltetrahydrofolic acid, and 5-formyltetrahydrofolic acid. Non-limiting examples of Vitamin B₁₂ include cyanocobalamin, hydroxycobalamin, and methylcobalamin. Non-limiting examples of Vitamin C include ascorbic acid, sodium ascorbate, sodium ascorbyl-2-monophosphate, calcium ascorbyl-2-monophosphate, magnesium ascorbyl-2-monophosphate, and sodium ascorbyl-2-polyphosphate.

ii. Microbicides

In another embodiment, the growth medium may include a microbicide. Suitable microbicides may include fungicides or bactericides. As will be appreciated by a skilled artisan, the choice of a fungicide or bactericide can and will vary depending upon the microalgae and the microbial target. Suitable non-limiting examples of fungicides and bactericides that may be used include the following: carbamate fungicides such as 3,3′-ethylenebis(tetrahydro-4,6-dimethyl-2H-1,3,5-thiadiazine-2-thione), zinc or manganese ethylenebis(dithiocarbamate), bis(dimethyldithiocarbamoyl)disulfide, zinc propylenebis(dithiocarbamate) bis(dimethyldithiocarbamoyl)ethylenediamine; nickel dimethyldithiocarbamate, methyl 1-(butylcarbamoyl)-2-benzimidazolecarbamate, 1,2-bis(3-methoxycarbonyl-2-thioureido)benzene, 1-isopropylcarbamoyl-3-(3,5-dichlorophenyl)hydantoin, potassium N-hydroxymethyl-N-methyldithiocarbamate and 5-methyl-10-butoxycarbonylamino-10,11-dehydrodibenzo(b,f)azepine; pyridine fungicides such as zinc bis(1-hydroxy-2(1H)pyridinethionate) and 2-pyridinethiol-1-oxide sodium salt; phosphorus fungicides such as O,O-diisopropyl S-benzylphosphorothioate and O-ethyl S,S-diphenyldithiophosphate; phthalimide fungicides such as N-(2,6-diethylphenyl)phthalimide and N-(2,6-diethylphenyl)-4-methylphthalimide; dicarboxylmide fungicides such as N-trichloromethylthio-4-cyclohexene-1,2-dicarboxylmide and N-tetrachloroethylthio-4-cyclohexene-1,2-dicarboxylmide; oxathine fungicides such as 5,6-dihydro-2-methyl-1,4-oxathine-3-carboxanilido-4,4-dioxide and 5,6-dihydro-2-methyl-1,4-oxathine-3-carboxanilide; naphthoquinone fungicide such as 2,3-dichloro-1,4-naphthoquinone, 2-oxy-3-chloro-1,4-naphthoquinone copper sulfate; pentachloronitrobenzene; 1,4-dichloro-2,5-dimethoxybenzene; 5-methyl-s-triazol(3,4-b)benzthiazole; 2-(thiocyanomethylthio)benzothiazole; 3-hydroxy-5-methylisooxazole; N-2,3-dichlorophenyltetrachlorophthalamic acid; 5-ethoxy-3-trichloromethyl-1-2,4-thiadiazole; 2,4-dichloro-6-(O-chloroanilino)-1,3,5-triazine; 2,3-dicyano-1,4-dithioanthraquinone; copper 8-quinolinate, polyoxine; validamycin; cycloheximide; iron methanearsonate; diisopropyl-1,3-dithiolane-2-iridene malonate; 3-allyloxy-1,2-benzoisothiazol-1,1-dioxide; kasugamycin; Blasticidin S; 4,5,6,7-tetrachlorophthalide; 3-(3,5-dichlorophenyl)-5-ethenyl-5-methyloxazolizine-2,4-dione; N-(3,5-dichlorophenyl)-1,2-dimethylcyclopropane-1,2-dicarboxylmide; S-n-butyl-5′-para-t-butylbenzyl-N-3-pyridyldithiocarbonylimidate; 4-chlorophenoxy-3,3-dimethyl-1-(1H,1,3,4-triazol-1-yl)-2-butanone; methyl-D,L-N-(2,6-dimethylphenyl)-N-(2′-methoxyacetyl)alaninate; N-propyl-N-[2-(2,4,6-trichlorophenoxy)ethyl]imidazol-1-carboxamide; N-(3,5-dichlorophenyl)succinimide; tetrachloroisophthalonitrile; 2-dimethylamino-4-methyl-5-n-butyl-6-hydroxypyrimidine; 2,6-dichloro-4-nitroaniline; 3-methyl-4-chlorobenzthiazol-2-one; 1,2,5,6-tetrahydro-4H-pyrrolo[3,2,1-i,j]quinoline-2-one; 3′-isopropoxy-2-methylbenzanilide; 1-[2-(2,4-dichlorophenyl)-4-ethyl-1,3-dioxorane-2-ylmethyl]-1H,1,2,4-triaz ol; 1,2-benzisothiazoline-3-one; basic copper chloride; basic copper sulfate; N′-dichlorofluoromethylthio-N,N-dimethyl-N-phenylsulfamide; ethyl-N-(3-dimethylaminopropyl)thiocarbamate hydrochloride; piomycin; S,S-6-methylquinoxaline-2,3-diyldithiocarbonate; complex of zinc and manneb; di-zinc bis(dimethyldithiocarbamate) ethylenebis (dithiocarbamate) and glyphosate. Additional suitable fungicides may include a chlorothalonil-based fungicide, a strobilurin-based fungicide, a triazole-based fungicide or a suitable combination of these fungicides. Non-limiting examples of suitable strobilurin-based fungicides include azoxystrobin, pyraclostrobin, or trifloxystrobin. Representative examples of triazole-based fungicides include myclobutanil, propiconazole, tebuconazol, and tetraconazole.

iii. Herbicides

In yet another embodiment, the growth medium may include an herbicide. As will be appreciated by a skilled artisan, the choice of an herbicide can and will vary depending upon the microalgae in the colony and the intended target of the herbicide. Non-limiting examples of suitable herbicides that may be used include imidazolinone, acetochlor, acifluorfen, aclonifen, acrolein, AKH-7088, alachlor, alloxydim, ametryn, amidosulfuron, amitrole, ammonium sulfamate, anilofos, asulam, atrazine, azafenidin, azimsulfuron, BAS 620H, BAS 654 00H, BAY FOE 5043, benazolin, benfluralin, benfuresate, bensulfuron-methyl, bensulide, bentazone, benzofenap, bifenox, bilanafos, bispyribac-sodium, bromacil, bromobutide, bromofenoxim, bromoxynil, butachlor, butamifos, butralin, butroxydim, butylate, cafenstrole, carbetamide, carfentrazone-ethyl, chlormethoxyfen, chloramben, chlorbromuron, chloridazon, chlorimuron-ethyl, chloroacetic acid, chlorotoluron, chlorpropham, chlorsulfuron, chlorthal-dimethyl, chlorthiamid, cinmethylin, cinosulfuron, clethodim, clodinafop-propargyl, clomazone, clomeprop, clopyralid, cloransulam-methyl, cyanazine, cycloate, cyclosulfamuron, cycloxydim, cyhalofop-butyl, 2,4-D, daimuron, dalapon, dazomet, 2,4DB, desmedipham, desmetryn, dicamba, dichlobenil, dichlorprop, dichlorprop-P, diclofop-methyl, difenzoquat metilsulfate, diflufenican, dimefuron, dimepiperate, dimethachlor, dimethametryn, dimethenamid, dimethipin, dimethylarsinic acid, dinitramine, dinocap, dinoterb, diphenamid, diquat dibromide, dithiopyr, diuron, DNOC, EPTC, esprocarb, ethalfluralin, ethametsulfuron-methyl, ethofumesate, ethoxysulfuron, etobenzanid, fenoxaprop-P-ethyl, fenuron, ferrous sulfate, flamprop-M, flazasulfuron, fluazifop-butyl, fluazifop-P-butyl, fluchloralin, flumetsulam, flumiclorac-pentyl, flumioxazin, fluometuron, fluoroglycofen-ethyl, flupoxam, flupropanate, flupyrsulfuron-methyl-sodiu-m, flurenol, fluridone, fluorochloridone, fluoroxypyr, flurtamone, fluthiacet-methyl, fomesafen, fosamine, glufosinate-ammonium, glyphosate, glyphosinate, halosulfuron-methyl, haloxyfop, HC-252, hexazinone, imazamethabenz-methyl, imazamox, imazapyr, imazaquin, imazethapyr, imazosuluron, imidazilinone, indanofan, ioxynil, isoproturon, isouron, isoxaben, isoxaflutole, lactofen, lenacil, linuron, MCPA, MCPA-thioethyl, MCPB, mecoprop, mecoprop-P, mefenacet, metamitron, metazachlor, methabenzthiazuron, methylarsonic acid, methyldymron, methyl isothiocyanate, metobenzuron, metobromuron, metolachlor, metosulam, metoxuron, metribuzin, metsulfuron-methyl, molinate, monolinuron, naproanilide, napropamide, naptalam, neburon, nicosulfuron, nonanoic acid, norflurazon, oleic acid (fatty acids), orbencarb, oryzalin, oxadiargyl, oxadiazon, oxasulfuron, oxyfluorfen, paraquat dichloride, pebulate, pendimethalin, pentachlorophenol, pentanochlor, pentoxazone, petroleum oils, phenmedipham, picloram, piperophos, pretilachlor, primisulfuron-methyl, prodiamine, prometon, prometryn, propachlor, propanil, propaquizafop, propazine, propham, propisochlor, propyzamide, prosulfocarb, prosulfuron, pyraflufen-ethyl, pyrazolynate, pyrazosulfuron-ethyl, pyrazoxyfen, pyributicarb, pyridate, pyriminobac-methyl, pyrithiobac-sodium, quinclorac, quinmerac, quinoclamine, quizalofop, quizalofop-P, rimsulfuron, sethoxydim, siduron, simazine, simetryn, sodium chlorate, STS system (sulfonylurea), sulcotrione, sulfentrazone, sulfometuron-methyl, sulfosulfuron, sulfuric acid, tar oils, 2,3,6-TBA, TCA-sodium, tebutam, tebuthiuron, terbacil, terbumeton, terbuthylazine, terbutryn, thenylchlor, thiazopyr, thifensulfuron-methyl, thiobencarb, tiocarbazil, tralkoxydim, tri-allate, triasulfuron, triaziflam, tribenuron-methyl, triclopyr, trietazine, trifluralin, triflusulfuron-methyl, and vernolate.

iv. Insecticides

In still another embodiment, the growth medium may include an insecticide. Non-limiting examples of suitable insecticides include the following: phosphoric insecticides such as O,O-diethyl O-(2-isopropyl-4-methyl-6-pyrimidinyl)phosphorothioate, O,O-dimethyl S-2-[(ethylthio)ethyl]phosphorodithioate, O,O-dimethyl O-(3-methyl-4-nitrophenyl)thiophosphate, O,O-dimethyl S-(N-methylcarbamoylmethyl)phosphorodithioate, O,O-dimethyl S-(N-methyl-N-formylcarbamoylmethyl) phosphorodithioate, O,O-dimethyl S-2-[(ethylthio)ethyl]phosphorodithioate, O,O-diethyl S-2-[(ethylthio)ethyl]phosphorodithioate, O,O-dimethyl-1-hydroxy-2,2,2-trichloroethylphosphonate, O,O-diethyl-O-(5-phenyl-3-isooxazolyl)phosphorothioate, O,O-dimethyl O-(2,5-dichloro-4-bromophenyl)phosphorothioate, O,O-dimethyl O-(3-methyl-4-methylmercaptophenyl)thiophosphate, O-ethyl O-p-cyanophenyl phenylphosphorothioate, O,O-dimethyl-S-(1,2-dicarboethoxyethyl)phosphorodithioate, 2-chloro-(2,4,5-trichlorophenyl)vinyldimethyl phosphate, 2-chloro-1-(2,4-dichlorophenyl)vinyldimethyl phosphate, O,O-dimethyl O-p-cyanophenyl phosphorothioate, 2,2-dichlorovinyl dimethyl phosphate, O,O-diethyl 0-2,4-dichlorophenyl phosphorothioate, ethyl mercaptophenylacetate O,O-dimethyl phosphorodithioate, S-[(6-chloro-2-oxo-3-benzooxazolinyl)methyl]O,O-diethyl phosphorodithioate, 2-chloro-1-(2,4-dichlorophenyl)vinyl diethylphosphate, O,O-diethyl O-(3-oxo-2-phenyl-2H-pyridazine-6-yl) phosphorothioate, O,O-dimethyl S-(1-methyl-2-ethylsulfinyl)-ethyl phosphorothiolate, O,O-dimethyl S-phthalimidomethyl phosphorodithioate, O,O-diethyl S-(N-ethoxycarbonyl-N-methylcarbamoylmethyl)phosphorodithioate, O,O-dimethyl S-[2-methoxy-1,3,4-thiadiazol-5-(4H)-onyl-(4)-methyl]dithiophosphate, 2-methoxy-4H-1,3,2-benzooxaphosphorine 2-sulfide, O,O-diethyl O-(3,5,6-trichloro-2-pyridyl)phosphorothiate, O-ethyl 0-2,4-dichlorophenyl thionobenzene phosphonate, S-[4,6-d]amino-s-triazine-2-yl-methyl]O,O-dimethyl phosphorodithioate, O-ethyl O-p-nitrophenyl phenyl phosphorothioate, O,S-dimethyl N-acetyl phosphoroamidothioate, 2-diethylamino-6-methylpyrimidine-4-yl-diethylphosphorothionate, 2-diethylamino-6-methylpyrimidine-4-yl-dimethylphosphorothionate, O,O-diethyl O-N-(methylsulfinyl)phenyl phosphorothioate, O-ethyl S-propyl O-2,4-dichlorophenyl phosphorodithioate and cis-3-(dimethoxyphosphinoxy)N-methyl-cis-crotone amide; carbamate insecticides such as 1-naphthyl N-methylcarbamate, S-methyl N-[methylcarbamoyloxy]thioacetoimidate, m-tolyl methylcarbamate, 3,4-xylyl methylcarbamate, 3,5-xylyl methylcarbamate, 2-sec-butylphenyl N-methylcarbamate, 2,3-dihydro-2,2-dimethyl-7-benzofuranylmethylcarbamate, 2-isopropoxyphenyl N-methylcarbamate, 1,3-bis(carbamoylthio)-2-(N,N-dimethylamino)propane hydrochloride and 2-diethylamino-6-methylpyrimidine-4-yl-dimethylcarbamate; and another insecticides such as N,N-dimethyl N′-(2-methyl-4-chlorophenyl)formamidine hydrochloride, nicotine sulfate, milbemycin, 6-methyl-2,3-quinoxalinedithiocyclic S,S-dithiocarbonate, 2,4-dinitro-6-sec-butylphenyl dimethylacrylate, 1,1-bis(p-chlorophenyl) 2,2,2-trichloroethanol, 2-(p-tert-butylphenoxy)isopropyl-2′-chloroethylsulfite, azoxybenzene, di-(p-chlorophenyl)-cyclopropyl carbinol, di[tri(2,2-dimethyl-2-phenylethyl)tin]oxide, 1-(4-chlorophenyl)-3-(2,6-difluorobenzoyl) urea and S-tricyclohexyltin O,O-diisopropylphosphorodithioate.

v. Ingredients for Fungus and Bacteria Growth

For embodiments in which fungus or bacteria is grown, the growth medium may further include carbon sources including but not limited to molasses, simple sugars including glucose, sucrose, dextrose, fructose, and maltose, and combinations thereof. The growth medium may also include a source of amino acids and nitrogen, including but not limited to yeast extract, bacto-peptone, ammonia, ammonium dihydrogen phosphate, and combinations thereof. The growth medium may further include other nutrient salts to supply the growing yeast with other vitamins and minerals. Non-limiting examples of other nutrient salts include biotin, vitamin B1, vitamin B6, calcium pantothenoate, calcium sulfate, inositol, copper, copper sulfate, zinc, zinc sulfate, iron, iron sulfate, potassium, potassium sulfate, magnesium, magnesium sulfate, potassium hydrozide, phosphoric acid, and combinations thereof.

### (d) Exemplary Growth Media

The particular composition of the growth media may be selected based on the nutritional needs of the species of unicellular organisms included in the colony. One exemplary growth medium suitable for growing diatoms and other coastal marine microalgae is f/2 medium, described in Guillard 1975, which is hereby incorporated by reference in its entirety. Table 1 summarizes the composition of f/2 medium:

TABLE 1
Composition of f/2 Medium

| | Concentration |
|---|---|
| Compound | (M) |
| NaNO₃ | 8.82 × 10⁻⁴ |
| NaH₂PO₄•H₂O | 3.62 × 10⁻⁵ |
| NaSiO₃•9H₂O | 1.06 × 10⁻⁴ |
| FeCl₃•6H₂O | 1.17 × 10⁻⁵ |
| Na₂EDTA•2H₂O | 1.17 × 10⁻⁵ |
| CuSO₄•5H₂O | 3.93 × 10⁻⁸ |
| Na₂MoO₄•2H₂O | 2.60 × 10⁻⁸ |
| ZnSO₄•7H₂O | 7.65 × 10⁻⁸ |
| CoCl₂•6H₂O | 4.20 × 10⁻⁸ |
| MnCl₂•4H₂O | 9.10 × 10⁻⁷ |
| Vitamin B₁ (thiamine HCl) | 2.96 × 10⁻⁷ |
| biotin | 2.05 × 10⁻⁹ |
| Vit B₁₂ | 3.69 × 10⁻¹⁰ |

Another exemplary growth medium suitable for growing fresh-water microalgal colonies is summarized in Table 2:

TABLE 2
Fresh-water Microalgal Growth Medium

| | Concentration |
|---|---|
| Compound | (mg/L) |
| NH₄Cl | 15 |
| MgCl₂•6H₂O | 12 |
| CaCl₂•2H₂O | 18 |
| MgSO₄•H₂O | 15 |
| KH₂PO₄ | 1.6 |
| FeCl₃•2H₂O | 0.08 |
| Na₂EDTA•2H₂O | 0.1 |
| H₃BO₃ | 0.185 |
| MnCl₂•4H₂O | 0.415 |
| ZnCl₂ | 3 × 10³ |
| CoCl₂•6H₂O | 1.5 × 10³ |
| CuCl₂•2H₂O | 1 × 10⁻⁵ |
| Na₂MoO₄•2H₂O | 7.0 × 10³ |
| NaNCO₃•2H₂O | 50 |

### IV. Growth Systems and Devices

The microalgal and other unicellular colonies of the present invention are grown in an aqueous growth medium, which provides essential macronutrients, micronutrients, and other compounds to the growing organisms. For embodiments in which microalgae is grown, the growth medium and microalgae are typically contained within either an open pond system or a photobioreactor that provides light and carbon dioxide to the growing microalgal colony. For embodiments in which bacteria or yeast are grown, the growth medium and growing cells are typically contained within a fermentor that provides appropriate growing conditions.

### (a) Open Pond System

An open pond system, as defined herein, includes a container that is typically relatively shallow in depth that further includes an exposed upper surface that is in direct contact with the surrounding atmosphere. The growth medium may be introduced to the open pond by methods including draining the pond and replacing with growth medium, or adding the compounds included in the growth medium to the existing water in the pond in amounts suitable transform the composition of the pond water into the desired growth medium composition. The microalgae may be introduced into the open pond system at a relatively low concentration and allowed to grow over a period of time sufficient to yield the desired cell density.

Carbon dioxide from the atmosphere dissolves into the growth medium via the exposed upper surface and diffuses to the microalgae for use in photosynthesis. Sunlight penetrating the exposed surface of the growth medium supplies the light energy for photosynthetic processes.

The open pond system may be completely exposed to the surrounding atmosphere. Alternatively, the open pond system may be partially enclosed using materials that are capable of transmitting adequate amounts of sunlight and fresh air to the exposed surface of the growth medium. For example, the open pond system may be sheltered by a transparent roof to prevent contamination of the growth medium by contaminants such as rainwater, pollen, fungal spores, and insects.

The open pond system may be a stationary body of water, or the open pond system may incorporate stirring devices such as paddle wheels or circulation pumps to mix the culture medium. In one embodiment, the open pond system may be an raceway pond in which the growth medium is directed by paddle wheels or circulation pumps through a continuous aquatic circuit containing a series of interconnected ponds.

### (b) Photobioreactor

A photobioreactor, as defined herein, is a closed system containing the microalgal colony. A photobioreactor typically includes a translucent container in which the microalgal colony is placed, along with a light source. The microalgae within the photobioreactor use the light from the light source in photosynthetic processes to actively grow and divide. Carbon dioxide for photosynthetic processes may be supplied to the microalgae passively by dissolving carbon dioxide gas into the growth medium via an exposed surface of the growth medium, or carbon dioxide may be actively supplied to the microalgae. For example, carbon dioxide gas may be bubbled through the growth medium by a gas line connected to a carbon dioxide gas source. Alternatively carbon dioxide may be supplied to the growth medium by the introduction of chemical reagents including acids such as hydrochloric acid and metal carbonates such as calcium carbonate that produce carbon dioxide via chemical reactions

Photobioreactors may be run in a batch mode, in which the microalgae is introduced into a container, and grown in the same container until harvest. The container may also incorporate stirring or mixing in order to enhance the uptake of nutrients to the microalgae. The container may be stirred continuously or periodically.

Alternatively, photobioreactors may operate in a continuous mode, in which fresh growth medium is introduced to the container of the photobioreactor continuously, and microalgae is continuously harvested. The rate of addition of the fresh growth medium and the rate of harvest of microalgae are approximately matched to prevent significant depletion or accrual of microalgae and growth medium within the photobioreactor.

The photobioreactor may additionally control other growth conditions such as the temperature and pH of the growth medium, and limit the presence of other microalgal species and/or other organisms.

### (c) Fermentor

A fermentor, as defined herein, is a closed system containing the bacteria or yeast colony. The bacteria or yeast cells are cultivated in a fermentation vessel. A fermentation vessel, as defined herein is any sterile container capable of holding the holding the bacteria or yeast as well as growth medium while the bacteria or yeast is fermenting. The fermentation vessel may be capable of conducting aerobic fermentation or anaerobic fermentation. Non-limiting examples of fermentation vessels known in the art include beakers, flasks, shake flasks, tanks, vats, fermentors, incubators, and bioreactors. In an embodiment, the fermentation vessel is capable of actively monitoring and adjusting various aspects of the fermentation environment including but not limited to agitation of the growth medium; dissolved gas concentration from the growth medium; temperature, pressure, or pH of the growth medium; nutrient concentrations in the growth medium; density o yeast cells in the growth medium, and combinations thereof.

In an embodiment, the fermentation vessel may be a fermentor with adjustable agitation speed, adjustable rate of flow of a mixture of gases comprising nitrogen, oxygen and carbon dioxide through the growth medium, and controllable rate of addition of nutrients. If the fermentation vessel is an anaerobic fermentation vessel, a mixture of gasses comprising nitrogen, carbon dioxide, and combinations thereof may flow through the growth culture to exclude oxygen from the growth culture. If the fermentation vessel is an aerobic fermentation vessel, air or pure oxygen may flow through the growth medium to induce aerobic metabolism by the fermenting bacteria or yeast cells.

V. Method of Increasing Growth, Lipid Content, and/or Protein Content of Microalgae

One aspect of the present invention provides a method of increasing the growth of a photosynthetic microalgal colony, described in section II(a), grown in an aqueous growth culture, described in section II(b). The method includes contacting the growth medium with an effective amount of a methionine compound, described in section I.

Another aspect of the present invention provides a method of increasing the lipid content of a photosynthetic microalgal colony, described in section II(a), grown in an aqueous growth culture, described in section II(b). The method includes contacting the growth medium with an effective amount of a methionine compound, described in section I.

In one embodiment, the growth of the microalgae is increased by at least 10% relative to the growth of the microalgae grown in the absence of the methionine compound. In other embodiments, the growth is increased by at least 20%, at least 30%, at least 40%, at least 60%, at least 80%, at least 100%, at least 150%, and at least 200% relative to the growth of the microalgae grown in the absence of the methionine compound. In an exemplary embodiment of the method, the growth is increased by at least 30% relative to the growth of the microalgae grown in the absence of the methionine compound.

In one embodiment, the lipid content of the microalgae is increased by at least 20% relative to the lipid content of the microalgae grown in the absence of the methionine compound. In other embodiments, the lipid content is increased by at least 30%, at least 50%, at least 75%, at least 100%, at least 150%, at least 200%, at least 300%, and at least 400% relative to the lipid content of the microalgae grown in the absence of the methionine compound. In an exemplary embodiment of the method, the lipid content of the microalgae is increased by at least 100% relative to the lipid content of the microalgae grown in the absence of the methionine compound.

### (a) Amount

The effective amount of the methionine compound may vary depending on a number of factors including but not limited to: the particular methionine compound contacted with the growth culture, the particular metal ion, if included, and the species of microalgae in the colony. In one embodiment, the effective amount of the methionine compound ranges from about 10⁻⁸ mg/ml to about 10⁻¹ mg/ml. In other embodiments, the effective amount of the methionine compound may range from about 10⁻⁸ mg/ml to about 10⁻⁷ mg/ml, from about 10⁻⁷ mg/ml to about 10⁻⁶ mg/ml, from about 10⁻⁶ mg/ml to about 10⁻⁵ mg/ml, from about 10⁻⁵ mg/ml to about 10⁻⁴ mg/ml, from about 10⁻⁴ mg/ml to about 10⁻³ mg/ml, from about 10⁻³ mg/ml to about 10⁻² mg/ml, and from about 10⁻² mg/ml to about 10⁻¹ mg/ml.

The methionine compound may be provided in the form of a dried powder or a concentrated aqueous solution. In one embodiment, the methionine compound may be dissolved into the growth medium.

### (b) Timing

The timing of addition of the methionine compound to be added to the growth medium depends on at least several factors. The methionine compound of the present invention, without being bound to any particular theory, may enhance growth at lower concentrations, and may also inhibit or terminate growth at higher concentrations, like many micronutrients of microalgae. Therefore, the concentration of the methionine compound in the growth medium may be limited to those amounts above a minimum concentration known to cause an increase in growth, and less than a maximum concentration known to either inhibit growth or known to be toxic to microalgae. In addition, the concentration of methionine compound in the growth medium may decrease with time as the growing microalgae deplete the dissolved methionine compound.

In one embodiment, the methionine compound may be contacted with the growth medium in a single dose. In another embodiment, the methionine compound may be contacted with the growth medium in a number of smaller discrete doses during the growth cycle of the microalgae, where the number of discrete doses may vary from 2 to about 100 doses. In this embodiment, the amount of methionine compound in each dose may be essentially the same for all doses, or the amount of methionine compound in each dose may vary between doses.

In yet another embodiment, the methionine compound may be introduced to the growth medium continuously at a constant rate. In still another embodiment, the methionine compound may be added continuously at a rate that varies according to the measured growth of the microalgae. For example, if measurements of the growth indicate that the microalgal growth is decreasing, the rate of addition of methionine compound may be increased. Methods of measuring the growth of the microalgal colony are described below.

### (c) Methods of Measuring Microalgal Growth, Lipid Content, and Protein Content

Microalgal growth may be expressed as any reasonable measure of cell density known in the art including the number of microalgal cells per unit volume of growth medium, the wet weight of the microalgal cells per unit volume of growth medium, and the dry weight of the microalgal cells per unit volume. In an exemplary embodiment, microalgal growth may be determined by estimating the density of the microalgal cells in the growth medium using devices known in the art including but not limited to: a haemocytometer, a microscope with a counting chamber, a spectrophotometer, a fluorometer, and a colorimeter. In another embodiment, microalgal growth may also be expressed as a growth rate, defined herein as the change in growth per unit time.

In yet another aspect, the growth of the microalgae may be expressed as a characteristic of the individual microalgal cells, including but not limited to the average lipid content of the microalgal cells, the average protein content of the microalgal cells, and combinations thereof. The average lipid contents of the microalgal cells may be estimated using known techniques including but not limited to fluorometric measurements of microalgal cells in which the lipids have been dyed with a lipid-indicating dye such as Nile Red (9-diethylamino-5H-benzophenoxazine-5-one). The average protein content of the microalgal cells may be estimated using devices and methods known in the art including but not limited to a spectrophotometer to measure the absorbance of light at a selected wavelength of microalgal cells in which the protein is dyed with a protein-indicating dye such as Coomassie Blue G dye.

In certain applications, the addition of methionine compounds to the growth medium may result in the inhibition of growth, reduction in the lipid and/or protein content, or elimination of the unicellular organisms in the growth medium of the colony. By way of specific example, and as presented in Example 3, the growth of Chlorella vulgaris, a fresh-water green microalgae, was inhibited by the addition of BIOX-C at a concentration of 10⁻² mg/mL to the growth medium.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Example 1

### Effect of Chelated Zinc on Growth of Dunaliella tertiolecta

To assess the efficacy of adding HMTBA-chelated zinc (BIOX-Z) to the growth medium on the growth of the microalgae Dunaliella tertiolecta, the following experiment was conducted. Dunaliella tertiolecta was maintained in stock cultures in 20 ml test tubes at a cell density of about 2.5-5.0×10⁴ cells/ml as seed cultures and maintained on a seven-day transfer cycle. The microalgae were inoculated into 1 L Pyrex glass Erlenmeyer flasks containing 250 ml of f/2 growth medium (Guillard 1975).

BIOX-Z was dissolved in distilled water and added at six different concentrations to each algal culture for final concentrations of 0 mg/ml (control), 10⁻⁶ mg/ml, 10⁻⁵ mg/ml, 10⁻⁴ mg/ml, 10⁻³ mg/ml and 10⁻² mg/ml. Each of the six test groups included 3 individual flasks.

The D. tertiolecta cells were grown in the flasks at a temperature of about 20° C. for a total of 18 days. Light was supplied to the cultures on a cycle of 12 hours of light and 12 hours of dark, at an intensity of 133-299 uEin/sec/cm² using a fluorescent light bulb bank (2-F20T12 Westinghouse cool white, Westinghouse Electric Corp., USA). Five samples from each of the test groups were taken each day, and the cell density of each sample was determined using a haemocytometer.

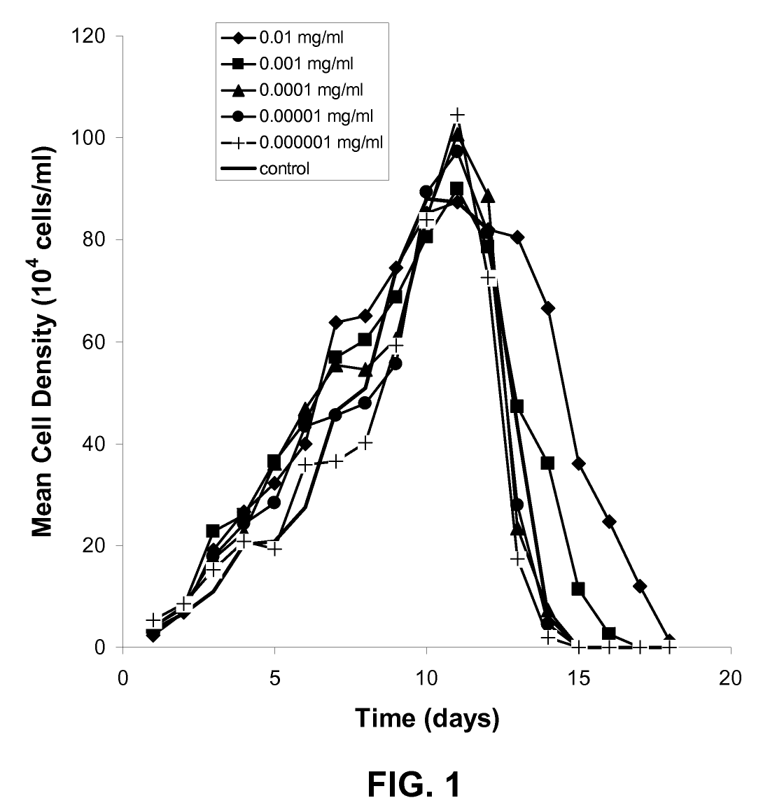

The mean concentration of D. tertiolecta cells cultured in the presence of the different concentrations of BIOX-Z is summarized in FIG. 1. The concentration of D. tertiolecta cells was enhanced when cultured in the presence of the BIOX-Z.

The results of this experiment demonstrated that the growth of Dunaliella tertiolecta was enhanced by the addition of BIOX-Z to the growth medium of the microalgae.

### Example 2

### Effect of Chelated Zinc on Growth of Nitzchia sp. Microalgae

To assess the efficacy of HMTBA-chelated zinc (BIOX-Z) on the growth of microalgae from the genus Nitzchia, the following experiment was conducted. Microalgae from the genus Nitzchia was maintained in stock cultures as described in Example 1. The microalgae were inoculated into 1 L flasks and varying amounts of BIOX-Z were added to the flasks as described in Example 1.

The Nitzchia cells were grown in the flasks as described in Example 1 for a total of 18 days. Samples were taken from the flasks each day to assess the cell density as described in Example 1.

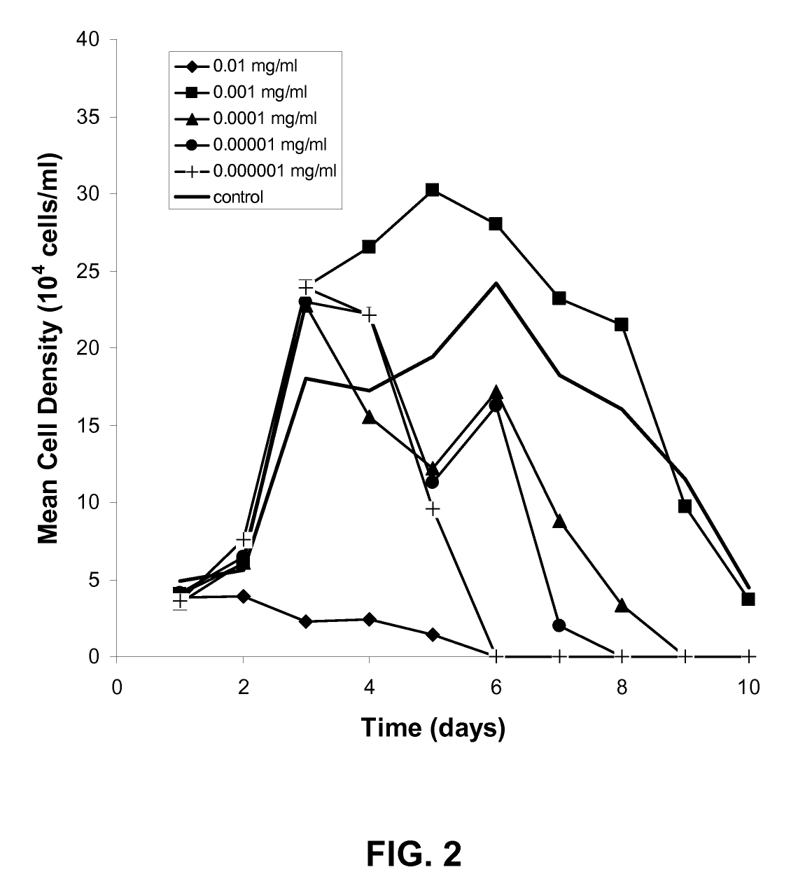

FIG. 2 summarizes the mean concentration of Nitzchia cells in the flasks as a function of time for each of the concentrations of BIOX-Z added to the growth media. The concentration of Nitzchia cells was enhanced when cultured in the presence of the chelated zinc compound at a concentration of 10⁻³ mg/ml.

The results of this experiment demonstrated that the growth of Nitzchia sp. microalgae increased by about 20%-60%, depending on the time of harvest, due to the addition of a chelated zinc compound to the growth medium of the microalgae.

### Example 3

### Effect of Chelated Micronutrients on the Growth of Chlorella vulgaris Microalgae

To assess the efficacy of chelated micronutrients on the growth of Chlorella vulgaris, a fresh-water green microalgae, the following experiment was conducted. Chlorella vulgaris microalgae were maintained in stock cultures as described in Example 1. The microalgae were inoculated at an initial concentration of about 10⁴ cells/ml into 250 ml conical flasks containing 100 ml of an algal growth medium having a composition described in Table 3.

TABLE 3
Composition of Algal Growth medium

| | Concentration |
|---|---|
| Compound | (mg/L) |
| NH₄Cl | 15 |
| MgCl₂•6 H₂O | 12 |
| CaCl₂•2 H₂O | 18 |
| MgSO₄•H₂O | 15 |
| KH₂PO₄ | 1.6 |
| FeCl₃•2H₂O | 0.08 |
| Na₂EDTA•2H₂O | 0.1 |
| H₃BO₃ | 0.185 |
| MnCl₂•4H₂O | 0.415 |
| ZnCl₂ | 3 × 10³ |
| CoCl₂•6H₂O | 1.5 × 10³ |
| CuCl₂•2H₂O | 1 × 10⁻⁵ |
| Na₂MoO₄•2H₂O | 7 × 10³ |
| NaHCO₃ | 50 |

Three different chelated micronutrient compositions were added to the growth medium of the flasks at six different concentrations to each algal culture for final concentrations of 0 mg/ml (control), 10⁻⁶ mg/ml, 10⁻⁶ mg/ml, 10⁻⁴ mg/ml, 10⁻³ mg/ml and 10⁻² mg/ml. The metal micronutrient compositions tested were Cu-HMTBA (BIOX-C), Zn-HMTBA (BIOX-Z), and Mn-HMTBA (BIOX-M). For each concentration of a particular micronutrient composition, three duplicate flasks were tested.

The flasks containing the added compounds were cultured in a chamber in which the temperature was maintained at 21° C.-25° C. In addition, the chamber provided continuous uniform illumination with a quantum flux of 0.72 10-20 photon/m²+/−20% in the spectral range of 400-700 nm (8000 flux). The contents of the flasks were shaken, stirred, or aerated during the experiment to facilitate the transfer of CO₂ to the microalgae.

Samples from each flask were taken after 2, 7, 12, 15, 18, 21, and 24 days of growth in the flasks and analyzed in quadruplicate to determine the cell density of the microalgae. The cell density was determined using a spectrophotometer at a wavelength setting of 450 nm and using a filtered growth medium sample as a blank. The sample volume analyzed was 100 μL per well.

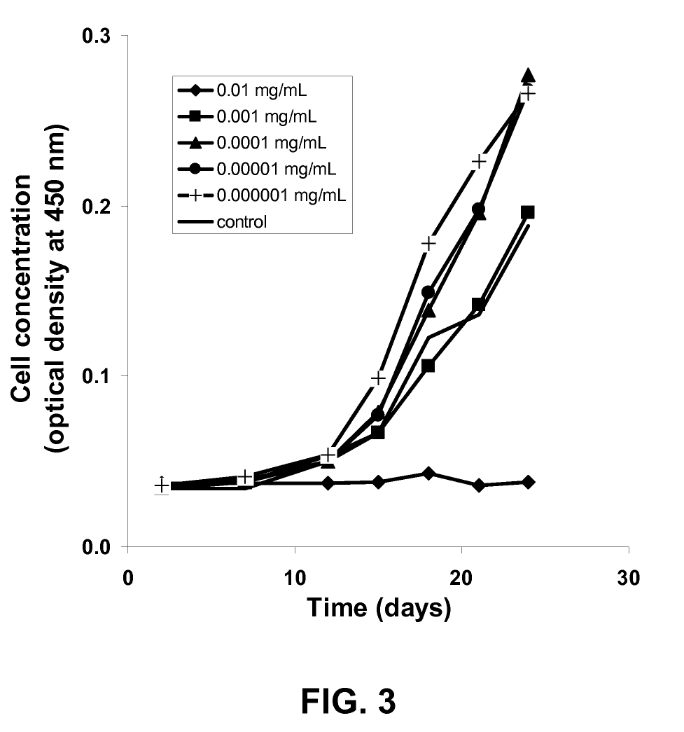

FIG. 3 summarizes the cell density of the Chlorella vulgaris colonies as a function of time for colonies grown in algal growth medium with 5 different concentrations of BIOX-C added. The growth of Chlorella vulgaris was enhanced by the addition of BIOX-C at concentrations of 10⁻⁶ mg/mL, 10⁻⁵ mg/mL, and 10⁻⁴ mg/mL. In addition, growth was inhibited by the addition of BIOX-C at a concentration of 10⁻² mg/mL, and the addition of BIOX-C at a concentration of 10⁻³ mg/mL had no effect on the growth of Chlorella vulgaris.

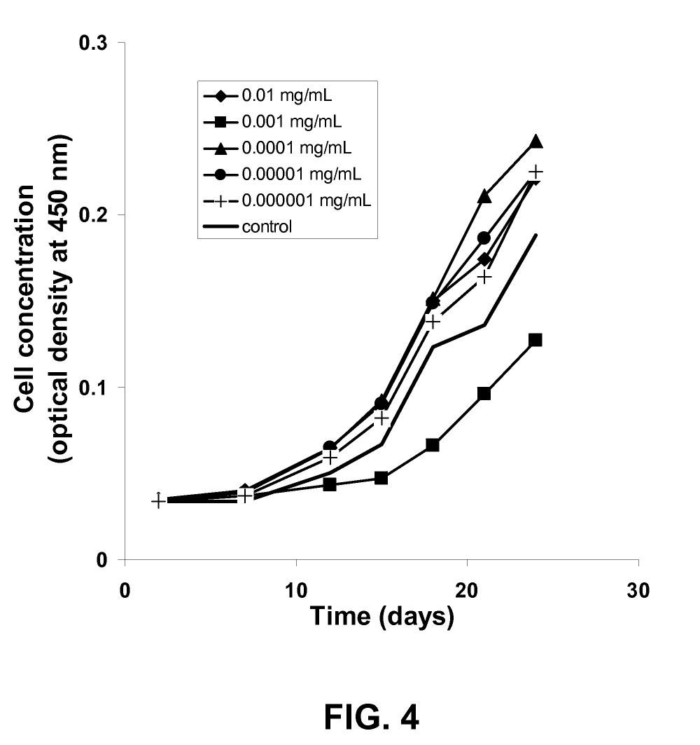

FIG. 4 summarizes the cell density of the Chlorella vulgaris colonies as a function of time for colonies grown in algal growth medium with 5 different concentrations of BIOX-M added. The growth of Chlorella vulgaris was enhanced by the addition of BIOX-M at concentrations of 10⁻⁶ mg/mL, 10⁻⁵ mg/mL, 10⁻⁴ mg/mL, and 10⁻² mg/mL. The growth of Chlorella vulgaris was inhibited by the addition of BIOX-M at a concentration of 10⁻³ mg/mL.

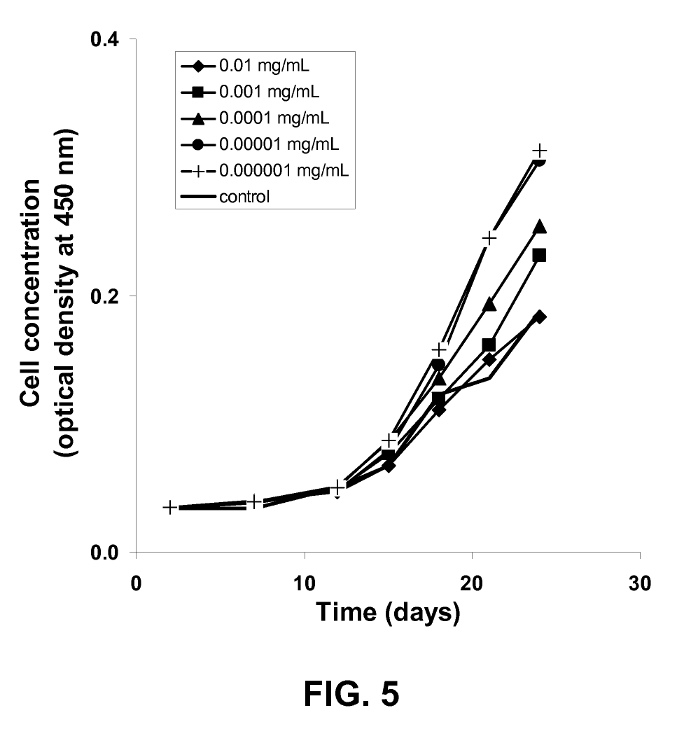

FIG. 5 summarizes the cell density of the Chlorella vulgaris colonies as a function of time for colonies grown in algal growth medium with 5 different concentrations of BIOX-Z added. The growth of Chlorella vulgaris was enhanced by the addition of BIOX-Z at concentrations of 10⁻⁶ mg/mL, 10⁻⁵ mg/mL, 10⁻⁴ mg/mL, and 10⁻³ mg/mL. The addition of BIOX-Z at a concentration of 10⁻² mg/mL had no effect on the growth of Chlorella vulgaris.

The results of this experiment demonstrated that culturing fresh-water microalgal cells in the presence of HMTBA-chelated micronutrients stimulated cell growth. The growth of algae was sensitive to the concentration of HMTBA-chelated micronutrients added to the algal growth medium, and growth was typically stimulated with concentrations of 10⁻⁴ or less.

### Example 4

### Effect of Chelated Micronutrients on Lipid Accumulation in Chlorella vulgaris Microalgae

To assess the efficacy of chelated micronutrients on the lipid content of Chlorella vulgaris, a fresh-water green microalgae, the following experiment was conducted. Chlorella vulgaris microalgae colonies were cultured in algal culture media containing added BIOX-C, BIOX-Z, and BIOX-M as described in Example 3.

Samples from each flask were taken after 9, 30, and 39 days of growth in the flasks and analyzed in triplicate to determine the lipid content of the microalgae cells. A fluorometric method was used determine cellular lipid accumulation. 4-mL culture samples were mixed with Nile Red (9-diethylamino-5H-benzophenoxazine-5-one) at a 100:1 volume ratio and allowed to stain for about seven minutes. Cytoplasmic lipids were then measured by the florescence intensity using a fluorometer (excitation 475 nm; emission 585 nm). The relative fluorescence for lipid content was obtained after subtraction of both the autofluorescence of algal cells and the self-fluorescence of the Nile Red dye from the uncorrected readings at 585 nm wavelengths.

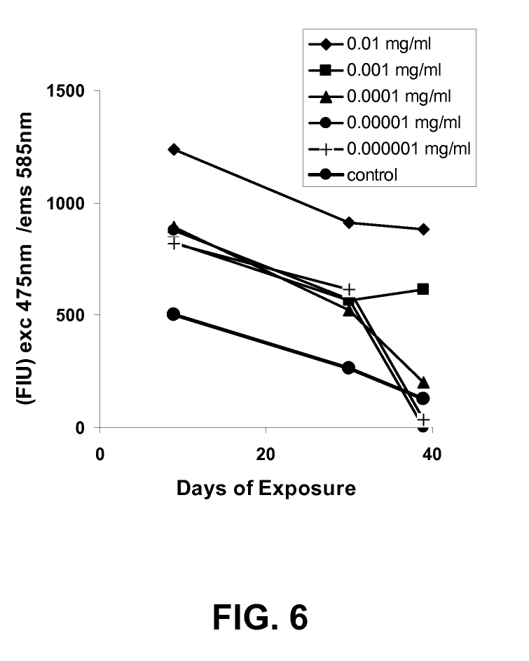

FIG. 6 summarizes the lipid content of the Chlorella vulgaris colonies as a function of time for colonies grown in algal growth medium with 5 different concentrations of BIOX-C added. The lipid content of the Chlorella vulgaris cells was enhanced by the addition of BIOX-C at all concentrations after 9 and 30 days of growth. However, after 39 of growth, only the microalgae grown with BIOX-C concentrations of 10⁻² mg/mL and 10⁻³ mg/mL had higher lipid content than the control colony.

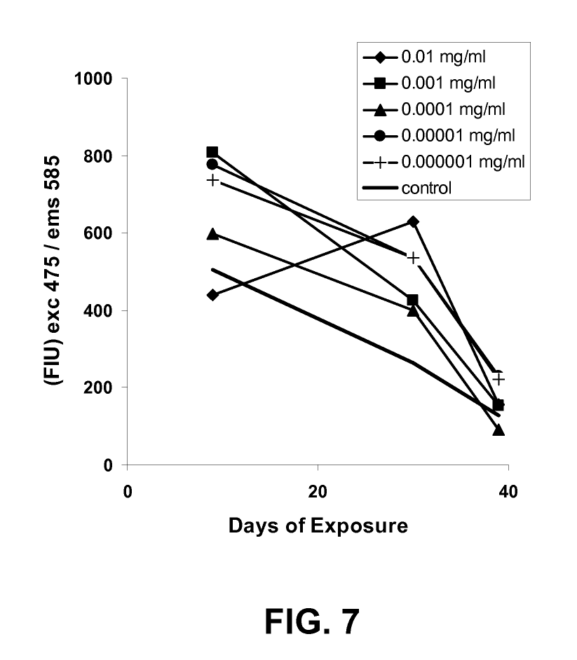

FIG. 7 summarizes the lipid content of the Chlorella vulgaris colonies as a function of time for colonies grown in algal growth medium with 5 different concentrations of BIOX-M added. The lipid content of the Chlorella vulgaris colonies after nine days were enhanced by the addition of BIOX-M at concentrations of less than 10⁻² mg/mL. After 30 days, the lipid content was enhanced by the addition of BIOX-M at all concentrations tested. After 39 days, the lipid content was enhanced to a lesser degree when cultivated in medium containing BIOX-M concentrations of 10⁻⁵ mg/mL and 10⁻⁶ mg/mL.

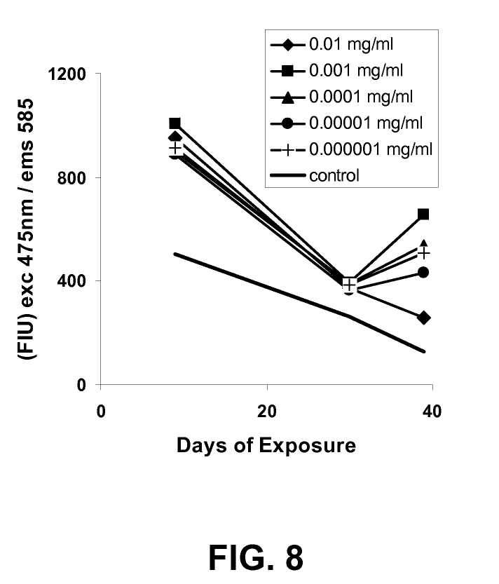

FIG. 8 summarizes the lipid content of the Chlorella vulgaris colonies as a function of time for colonies grown in algal growth medium with 5 different concentrations of BIOX-Z added. The lipid content of the Chlorella vulgaris cells was enhanced by the addition of BIOX-Z at all concentrations through the entire period of growth. The lipid content was most enhanced by growth in a medium containing BIOX-Z at a concentration of 10⁻³ mg/mL.

The results of this experiment demonstrated that the addition of BIOX-C, BIOX-M, and BIOX-Z enhanced the lipid content of Chlorella vulgaris cells. The degree of enhancement of lipid content was sensitive to the concentration of BIOX-C, BIOX-M, or BIOX-Z added. Typically, an initially large enhancement of cell lipid content after 9 days in culture was followed by an attenuation of the enhancement of the lipid accumulation in the algal cells after 39 days. The addition of BIOX-C at a concentration of 10⁻² mg/mL to the algal growth medium caused the largest enhancement of microalgal lipid content of all compositions tested.

### Example 5

### Effect of Chelated Micronutrients on Protein Content of Chlorella vulgaris Microalgae

To assess the efficacy of chelated micronutrients on the protein content of Chlorella vulgaris, a fresh-water green microalgae, the following experiment was conducted. Chlorella vulgaris microalgae colonies were cultured in algal culture media containing added BIOX-C, BIOX-Z, and BIOX-M as described in Example 3.

Samples from each flask were taken after 2, 7, 12, 15, 21, and 30 days of growth in the flasks and analyzed in triplicate to determine the protein content of the microalgae cells. The protein contents of the microalgal colonies were determined by using a spectrophotometer to measure microalgal cells in which the proteins were marked with a dye. 0.04-mL culture samples were gently mixed with 2 ml of Coomassie Blue G dye in a 4 mL plastic cuvette. Cytoplasmic proteins were then measured by measuring the absorbance of the dyed sample at a wavelength of 595 nm using a fluorometer.

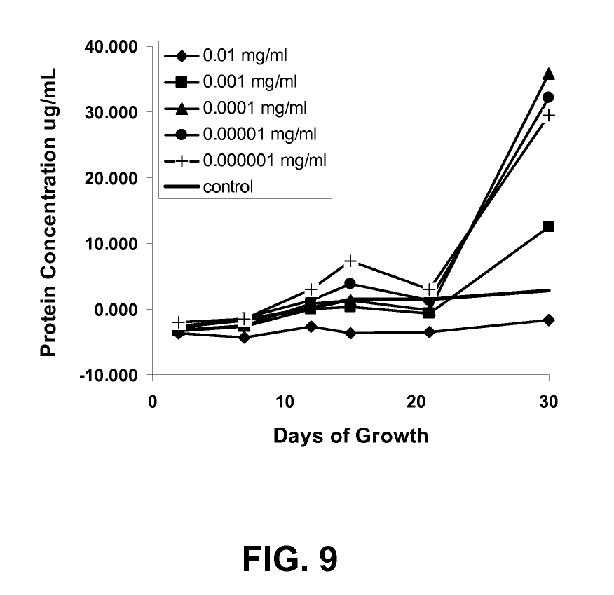

FIG. 9 summarizes the protein content of the Chlorella vulgaris colonies as a function of time for colonies grown in algal growth medium with 5 different concentrations of BIOX-C added. The protein content of the Chlorella vulgaris cells was enhanced by the addition of BIOX-C at all concentrations except 0.01 mg/mL at twelve days of growth or longer. After thirty days of growth, the protein content had increased in a dose-dependent manner for BIOX-C concentrations of 0.000001-0.0001 mg/ml to protein contents that were between about 10-12 times the control protein concentrations. The protein content of the Chlorella vulgaris cells cultured for thirty days in the presence of BIOX-C at a concentration 0.001 mg/mL was about 4.5 times the control protein content. BIOX-C at a concentration of 0.01 mg/mL inhibited the protein content of the microalgae cells to less than the protein content of the control cells.

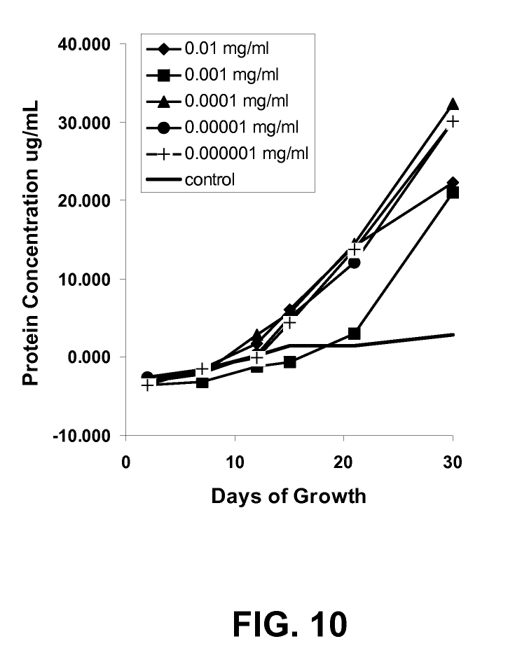

FIG. 10 summarizes the protein content of the Chlorella vulgaris colonies as a function of time for colonies grown in algal growth medium with 5 different concentrations of BIOX-M added. The protein contents of the Chlorella vulgaris colonies were enhanced by the addition of BIOX-M at all concentrations after 21 days or more of growth. After thirty days of growth, the protein content of the cells grown in the presence of BIOX-M at concentrations of 0.000001-0.0001 mg/mL had increased to about 10-12 times the control protein content. The cells grown in the presence of BIOX-M at concentrations of 0.001 and 0.01 mg/mL had protein contents that were 7-8 times the control protein content.

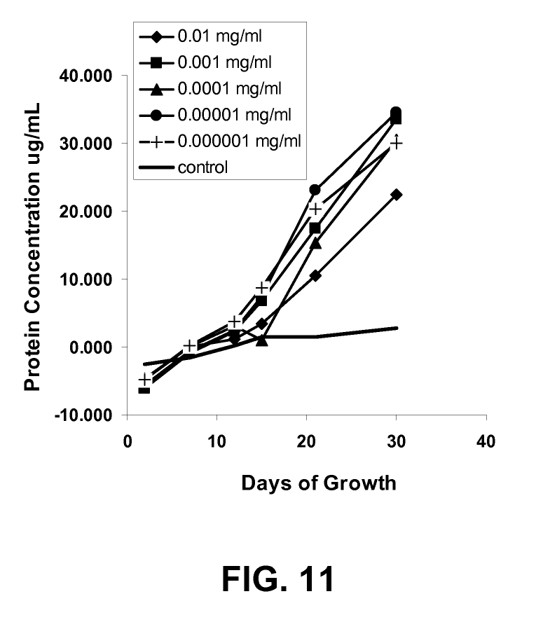

FIG. 11 summarizes the protein content of the Chlorella vulgaris colonies as a function of time for colonies grown in algal growth medium with 5 different concentrations of BIOX-Z added. The protein content of the Chlorella vulgaris cells was enhanced by the addition of BIOX-Z at all concentrations through the entire period of growth. The protein content was most enhanced by growth in a medium containing BIOX-Z at a concentration of 10⁻⁵ mg/mL. At this concentration of BIOX-Z, the microalgae cells had a protein content that was over twelve times the control protein content after 30 days of growth.

The results of this experiment demonstrated that the addition of BIOX-C, BIOX-M, and BIOX-Z enhanced the protein content of Chlorella vulgaris cells. The degree of enhancement of protein content was sensitive to the concentration of BIOX-C, BIOX-M, or BIOX-Z added. Typically, the enhancement of cell protein content did not occur until after about 12 days of exposure to BIOX-C, BIOX-M, or BIOX-Z. The addition of BIOX-C at a concentration of 10⁻⁴ mg/mL to the algal growth medium caused the largest enhancement of microalgal protein content of all compositions tested, corresponding to a protein content that was about 12.8 times the control protein content after thirty days of growth.

### REFERENCES

Guillard, R. R. L. 1975. Culture of phytoplankton for feeding marine invertebrates. Pp. 26-60 in Smith W. L. and Chanley M. N. (Eds.), Culture of Marine Invertebrate Animals. Plenum Press, NY, N.Y., USA.

What is claimed is:

1. A method for increasing the growth of a photosynthetic microalgal cell of a colony grown in an aqueous growth medium, the method comprising contacting the growth medium with a compound of Formula (III) or with a metal salt or metal chelate of a compound of Formula (III): 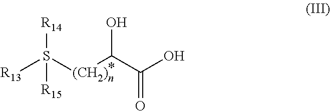
wherein:
* is a chiral carbon;
n is an integer from 1 to 3;
R₁₃ is methyl or ethyl; and
R₁₄ and R₁₅ are independently oxygen or hydrogen; wherein the growth of the microalgae is increased by at least 10% compared to the growth of microalgae grown without the compound.

2. The method of claim 1, wherein the compound of Formula (III) is 2-hydroxy-4(methylthio)butanoic acid.

3. The method of claim 1, wherein the compound is a metal chelate comprising a compound of Formula (III) together with a metal ion.

4. The method of claim 1, wherein the compound is a metal salt comprising a compound of Formula (III) together with a metal ion.

5. The method of claim 3, wherein the metal ion is chosen from boron ions, calcium ions, cobalt ions, copper ions, iron ions, manganese ions, molybdenum ions, nickel ions, and zinc ions.

6. The method of claim 1, wherein the compound is a metal chelate comprising 2-hydroxy-4(methylthio)butanoic acid and copper.

7. The method of claim 6, wherein an effective amount of the compound comprises a concentration in the growth medium ranging from about 10⁻⁸ mg/ml to about 10⁻¹ mg/ml.

8. The method of claim 1, wherein the microalgae is chosen from Charophyta, Chlorophyta, Diatoms, Rhodophyta, Cryptophyta, Chlorarachniophyta, Haptophyta, Euglenophyta, Heterokonta, and combinations thereof.

9. The method of claim 1, wherein the growth medium comprises an effective amount of at least one macronutrient chosen from nitrogen, phosphorus, potassium, and combinations thereof.

10. The method of claim 1, wherein the growth of the microalgae is increased by at least 30%, by at least 50%, by at least 75% or by at least 100% compared to the growth of microalgae grown without the compound.

11. A method for increasing a lipid content in a photosynthetic microalgal cell of a colony grown in an aqueous growth medium, the method comprising contacting the growth medium with a compound of Formula (III) or with a metal salt or metal chelate of a compound of Formula (III): 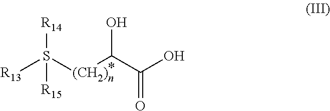
wherein:
* is a chiral carbon;
n is an integer from 1 to 3;
R₁₃ is methyl or ethyl; and
R₁₄ and R₁₅ are independently oxygen or hydrogen; wherein the amount of lipid contained in the photosynthetic microalgal cell is increased by at least 50% compared to the amount of lipid contained in the photosynthetic microalgal cell grown without the compound.

12. The method of claim 11, wherein the compound of Formula (III) is 2-hydroxy-4(methylthio)butanoic acid.

13. The method of claim 11, wherein the compound is a metal chelate comprising a compound of Formula (III) together with a metal ion.

14. The method of claim 11, wherein the compound is a metal salt comprising a compound of Formula (III) together with a metal ion.

15. The method of claim 13, wherein the metal ion is chosen from boron ions, calcium ions, cobalt ions, copper ions, iron ions, manganese ions, molybdenum ions, nickel ions, and zinc ions.

16. The method of claim 11, wherein the compound is a metal chelate comprising 2-hydroxy-4(methylthio)butanoic acid and copper.

17. The method of claim 16, wherein an effective amount of the compound comprises a concentration in the growth medium ranging from about 10⁻⁸ mg/ml to about 10⁻¹ mg/ml.

18. The method of claim 11, wherein the microalgae is chosen from Charophyta, Chlorophyta, Diatoms, Rhodophyta, Cryptophyta, Chlorarachniophyta, Haptophyta, Euglenophyta, Heterokonta, and combinations thereof.

19. The method of claim 11, wherein the growth medium comprises an effective amount of at least one macronutrient chosen from nitrogen, phosphorus, potassium, and combinations thereof.

20. The method of 11, wherein the amount of lipid contained in the photosynthetic microalgal cell is increased by at least 100%, at least 150%, or at least 200% compared to the amount of lipid contained in the photosynthetic microalgal cell grown without the compound.

21. A method for increasing a protein content in a photosynthetic microalgal cell of a colony grown in an aqueous growth medium, the method comprising contacting the growth medium with a compound of Formula (III) or with a metal salt or metal chelate of a compound of Formula (III): 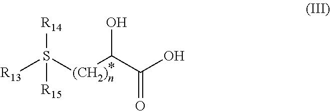
wherein:
* is a chiral carbon;
n is an integer from 1 to 3;
R₁₃ is methyl or ethyl; and
R₁₄ and R₁₅ are independently oxygen or hydrogen; wherein the amount of protein contained in the photosynthetic microalgal cell is increased by at least 500% compared to the amount of protein contained in the photosynthetic microalgal cell grown without the compound.

22. The method of claim 21, wherein the compound of Formula (III) is 2-hydroxy-4(methylthio)butanoic acid.

23. The method of claim 21, wherein the compound is a metal chelate comprising a compound of Formula (III) together with a metal ion.

24. The method of claim 21, wherein the compound is a metal salt comprising a compound of Formula (III) together with a metal ion.

25. The method of claim 23, wherein the metal ion is chosen from boron ions, calcium ions, cobalt ions, copper ions, iron ions, manganese ions, molybdenum ions, nickel ions, and zinc ions.

26. The method of claim 21, wherein the compound is a metal chelate comprising 2-hydroxy-4(methylthio)butanoic acid and copper.

27. The method of claim 26, wherein an effective amount of the compound comprises a concentration in the growth medium ranging from about 10⁻⁸ mg/ml to about 10⁻¹ mg/ml.

28. The method of claim 21, wherein the microalgae is chosen from Charophyta, Chlorophyta, Diatoms, Rhodophyta, Cryptophyta, Chlorarachniophyta, Haptophyta, Euglenophyta, Heterokonta, and combinations thereof.

29. The method of claim 21, wherein the growth medium comprises an effective amount of at least one macronutrient chosen from nitrogen, phosphorus, potassium, and combinations thereof.

30. The method of claim 21, wherein the amount of protein contained in the photosynthetic microalgal cell is increased by at least 750%, at least 1000%, at least 1250%, at least 1500%, or by greater than 2000% compared to the amount of protein contained in the photosynthetic microalgal cell grown without the compound.
